(19) United States
(12) Patent Application Publication
(10) Pub. No.: US 2024/0050440 A1
(43) Pub. Date: Feb. 15, 2024

(54) THERAPEUTIC TARGETS AND AGENTS FOR THE TREATMENT OF TRIOSEPHOSPHATE ISOMERASE (TPI) DEFICIENCY

(71) Applicant: University of Pittsburgh- Of The Commonwealth System of Higher Education, Pittsburgh, PA (US)

(72) Inventors: Michael John Palladino, Pittsburgh, PA (US); Stacy Lynn Hrizo, Cranberry Township, PA (US); Andreas Vogt, Pittsburgh, PA (US)

(73) Assignee: University of Pittsburgh - Of The Commonwealth System of Higher Education, Pittsburgh, PA (US)

(21) Appl. No.: 18/269,260

(22) PCT Filed: Dec. 30, 2021
(86) PCT No.: PCT/US2021/065639

§ 371 Date: Jun. 22, 2023

Related U.S. Application Data
(60) Provisional application No. 63/132,770, filed on Dec. 31, 2020.
(60) Provisional application No. 63/132,787, filed on Dec. 31, 2020.

(51) Int. Cl. A61K 31/5377; A61P 25/00; A61K 31/357; C12N 9/90; G01N 33/50; A61K 31/05; G01N 21/64

(52) U.S. Cl. CPC A61K 31/5377; A61P 25/00; A61K 31/357; C12N 9/90; G01N 33/5008; A61K 31/05; G01N 21/6486; C12Y 503/01001

(57) ABSTRACT

Triosephosphate isomerase deficiency (TPI Df) is a devastating childhood degenerative disease for which there are currently no treatments. Pathogenesis of this disease is driven by mutations that destabilize the TPI protein. A genome-wide RNAi screen in Drosophila to identify regulators of TPI stability is described. The screen identified 25 proteins that are critical to TPI stability, each of which has a human ortholog. Methods of promoting TPI protein stability and treating TPI Df in a subject by administering a therapeutically effective amount of an agent that inhibits expression or activity of one of the identified regulators are described. Also described is a method for the identification of agents capable of promoting stability of mutant TPI. Methods for treating a subject who has TPI Df by administering an agent that promotes stability of mutant TPI is also described.

### CROSS REFERENCE TO RELATED APPLICATIONS

This application claims the benefit of U.S. Provisional Application No. 63/132,787, filed Dec. 31, 2020 and U.S. Provisional Application No. 63/132,770, filed Dec. 31, 2020, which are herein incorporated by reference in their entirety.

### FIELD

This disclosure concerns the identification of therapeutic targets in the protein quality control (PQC) pathway that modulate turnover of a mutant form of the triosephosphate isomerase (TPI) enzyme. This disclosure further concerns an assay to identify agents that inhibit degradation of mutant TPI protein and use of the agents in the treatment of TPI deficiency.

### ACKNOWLEDGMENT OF GOVERNMENT SUPPORT

This invention was made with government support under grant numbers AG059385, GM103369, HD105311 and HD104346 awarded by the National Institutes of Health. The government has certain rights in the invention.

### BACKGROUND

Triosephosphate isomerase (TPI) is a glycolytic enzyme that converts dihydroxyacetone phosphate to glyceraldehyde-3-phosphate. Numerous mutations affecting this enzyme are associated with a devastating recessive degenerative neuromuscular disorder called TPI deficiency (TPI Df) for which there are currently no treatments. Individuals homozygous for the “common” TPI^{E105D} allele or compound heterozygous with various alleles are affected. Patients typically exhibit childhood-onset hemolytic anemia, reduced immune function, progressive neuromuscular degeneration and premature death. Data obtained with patient cells suggests that the missense mutations in the TPI gene produce enzymes that are less stable, resulting in reduced TPI protein. There are currently no treatments for patients with TPI Df, thus a need exists for the identification of novel pharmacological targets to enable development of treatments for this disease.

A Drosophila strain has been isolated with a mutation in TPI called sugarkill that models human TPI Df. TPI^{sugarkill} is recessive and in homozygotes causes TPI protein instability associated with progressive locomotor impairment, neurodegeneration and early death. Previous studies on these flies found that the TPI^{sugarkill} protein is bound by the molecular chaperones Hsp70 and Hsp90 and is subsequently degraded by the proteasome. In addition, this instability can be modulated pharmacologically by targeting the molecular chaperone Hsp90 or the proteasome, leading to an increase in mutant protein, which improves animal phenotypes and survival. It has been speculated that stabilizing mutant TPI protein could be a viable therapeutic approach for TPI Df. Although there are several known drugs that modulate HSP70, HSP90 and the proteasome, all currently available drugs are exceptionally toxic and their clinical significance centers on their established toxicity and ability to sensitize cells to chemotherapies. The proteasome together with promiscuous chaperones such as HSP70 and HSP90 regulate much of the proteome suggesting their inhibitors may not be viable for use as chronic therapies.

The protein quality control (PQC) pathway in the endoplasmic reticulum has been well studied with a variety of endogenous substrates). However, PQC regulators within the cytosolic compartment are less well defined, especially for substrates that are not prone to aggregation. The TPI^{sugarkill} protein is a soluble, functional, non-aggregation prone, cytoplasmic protein that is targeted for degradation by the proteasome, Both TPI^{sgk} and TPI^{E105D} have alterations affecting their dimer interface and both proteins are temperature-sensitive and possibly thermolabile by an incompletely understood mechanism leading to increased rates of degradation when cells are exposed to heat stress. Patient fibroblasts with the severe TPI Df allele TPI^{R189Q} exhibit extremely low TPI levels, and the patient has a clinical history of increasing severity of symptoms coincident with infections, suggesting the patient's severe symptoms may be febrile in origin.

### SUMMARY

The pathogenesis of TPI Df is associated with destabilizing mutations affecting the TPI protein that lead to a reduction in the amount of TPI protein present in patient cells. A need exists for the identification of genes involved in promoting stability of the mutant TPI enzyme, and for a high-throughput method of identifying candidate therapeutic agents that promote stability of mutant TPI protein, which could be used for the treatment of TPI Df. Disclosed herein is the identification of therapeutic targets in the protein quality control (PQC) pathway that modulate turnover of mutant TPI protein resulting from a missense mutation in the human TPI gene (TPI^{E105D}) Also disclosed is a high-throughput method for identifying agents that promote stability of the mutant form of TPI.

Provided herein is a method of treating TPI Df in a subject. In some embodiments, the method includes administering to the subject a therapeutically effective amount of an agent that inhibits expression or activity of aconitase 1 (ACO1), actin related protein 3 (ACTR3), anaphase promoting complex subunit 10 (ANAPC10), anti-silencing function 1A histone chaperone (ASF1A), cell division cycle 34, ubiquitin conjugating enzyme (CDC34), carboxypeptidase B1 (CPB1), crystallin alpha A (CRYAA), cullin 3 (CUL3), dynein axonemal assembly factor 4 (DNAAF4), DnaJ heat shock protein family (Hsp40) member B11 (DNAJB11), DnaJ heat shock protein family (Hsp40) member C2 (DNAJC2), DnaJ heat shock protein family (Hsp40) member C15 (DNAJC15), endoplasmic reticulum protein 29 (ERP29), GTP binding protein 6 (GTPBP6), heat shock protein family B (small) member 1 (HSPB1), HECT, UBA and WWE domain containing E3 ubiquitin protein ligase 1 (HUWE1), MPV17 mitochondrial inner membrane protein like 2 (MPV17L2), proteasome 20S subunit alpha 7 (PSMA7), SEC61 translocon subunit gamma (SEC61G), translocase of outer mitochondrial membrane 22 (TOMM22), ubiquitin conjugating enzyme E2 B (UBE2B), ubiquitin conjugating enzyme E2 Q1 (UBE2Q1), ubiquitin specific peptidase 14 (USP14), ubiquitin specific peptidase 16 (USP16), or ubiquitin specific peptidase 45 (USP45). In some examples, the agent inhibits expression of the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 gene. In other examples, the agent inhibits activity of a protein encoded by the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 gene.

Also provided is a method of promoting stability of a mutant form of the human TPI protein in human cells. In some embodiments, the method includes contacting the cells with an effective amount of an agent that inhibits expression or activity of ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45. In some examples, the method is an in vitro method. In other examples, the method is an in vivo method.

Further provided herein is a method for identifying an agent that promotes stability of a mutant form of the human TPI protein. In some embodiments, the method includes culturing in a cell culture vessel cells stably expressing a fusion protein comprising the mutant TPI protein and a fluorescent protein; contacting the cells with a candidate agent; and measuring fluorescence intensity of the cells. An increase in fluorescence intensity of the cells compared to control cells cultured in the absence of the candidate agent, identifies the candidate agent as an agent that promotes stability of the mutant TPI protein. The mutant TPI protein can possess any destabilizing amino acid substitution resulting from mutation of the TPI gene, such as a mutation listed in Table 1. In specific non-limiting examples, the mutant TPI protein has a glutamic acid to aspartic acid substitution at position 105 (TPI^{E105D}) Also provided is a method of treating a subject who has TPI Df by administering to the subject a therapeutically effective amount of an agent identified by the method disclosed herein.

Also provided is a method of treating TPI Df in a subject by administering to the subject a therapeutically effective amount of an agent that promotes stability of a mutant form of the human TPI protein. In some embodiments, the mutant TPI protein results from a mutation listed in Table 1. In some examples, the mutant TPI protein has a glutamic acid to aspartic acid substitution at position 105 (TPI^{E105D}).

The foregoing and other objects and features of the disclosure will become more apparent from the following detailed description, which proceeds with reference to the accompanying figures.

### BRIEF DESCRIPTION OF THE DRAWINGS

FIGS. 1A-1C: RNAi targeting of select factors resulted in stabilized levels of TPI^{sugarkill}. The no RNAi samples reared at 25° C. (0 h control) and 29° C. for 48h were used to determine the relative expected instability of TPI^{sugarkill} The stability of TPI^{sugarkill} in the RNAi expressing lines was compared to relative TPI^{sugarkill} levels in the RNAi 29° C. samples on the same blot. For each R line, n=4 sets of Western blots were performed. The rescued lines were all significantly different than the 29° C. TPI^{sugarkill} protein levels by a Student's t-test (labelled 2 and 3 in FIG. 1C, p<0.05). A Bonferroni post-test was performed (labelled 3 in FIG. 1C, p<0.00011). The samples that were initially identified as potential factors involved in TPI^{sugarkill} turnover were selected for a second round of screening (labelled 2 and 3 in FIG. 1C). The identity of the factors shown in FIG. 1A and FIG. 1B are as follows: 424 (CG4599, TPR 2), 150 (CG8937, Hsp70), 275 (CG12161, proteasome 132 subunit 2).

FIG. 2: A secondary round of screening identified 25 potential regulators of TPI^{sugarkill} degradation. The no RNAi samples reared at 25° C. (0 h control) and 29° C. for 48h were used to determine the relative expected instability of TPI^{sugarkill} The stability of TPI^{sugarkill} in the RNAi expressing lines was compared to relative TPI^{sugarkill} levels in the RNAi 29° C. samples on the same blot. For each RNAi line, n=4 sets of Western blots were performed. Samples were compared to the no RNAi 48h 29° C. control set by One-Way ANOVA with a Dunnett's Post Test (2: p>0.05, 3: p<0.05).

FIG. 3: Chaperones and protein degradation regulators were enriched in the hits from both rounds of screening. A STRING network was developed by evaluating the genes identified in 2 rounds of Western blot screening of RNAi samples. The interactions were assessed as follows: experimental evidence, database evidence, co-expression data, text mining data and co-occurrence. Thickness of lines indicates strength of evidence for interaction.

FIG. 4. TPI^{sugarkill} is polyubiquitinated. Flies treated with MG132 for 24 hours and incubated at either 25° C. or 29° C. were homogenized and TPI^{sugarkill} was immunoprecipitated with anti-TPI. A bead only negative control was included. The characteristic polyubiquitin smear is visible in the samples with immunoprecipitated TPI^{sugarkill}.

FIGS. 5A-5D: Knock down of Cullin 3, HIP and Hsp90 with p-element disruption results in increased TPI^{sugarkill} protein levels that correlate to extended lifespan and reduced bang sensitivity in TPI^{sugarkill} homozygotes. P-elements were used to disrupt the expression of the Cullin 3, HIP and Hsp90 genes. Their flies were crossed to be homozygous for both the p-element disruption and the TPI^{sugarkill} allele. (FIG. 5A) Western blots were completed following incubation of flies at 25° C. for 5 days and compared to a TPI^{sugarkill} homozygote control incubated at the same time. Student's t-tests were used to determine significance (* p<0.05). Error bars are SEM. (FIG. 5B) Mechanical stress sensitivity assays were completed on flies reared at 25° C. Analysis of HIP (n=50), Cullin 3 (n=30) and Hsp90 (n=25) is shown. Average time to recovery was calculated and compared to a TPI^{sugarkill} homozygote control using PRISM software for each p-element line (* p<0.05, **p<0.01 and ***p<0.001). Error bars shown are SEM. (FIGS. 5C-5D) Lifespans were completed in triplicate with approximately 25 flies per vial at 25° C. Survival curves and median lifespan were compared to a TPI^{sugarkill} homozygote control using PRISM software for each p-element line.

FIGS. 6A-6C: Overexpression of RAC-associated HSP40 (Zuo1) results in TPI^{sugarkill} destabilization and reduced lifespan. (FIG. 6A) Western blots were conducted following a 29° C. temperature shift for 48h on TPI^{sugarkill} homozygotes with and without an extra copy of Zuo1-GFP for increased expression. Densitometry was completed using Image J and the results were analyzed for significance by Student's t-test (n=7, P<0.0001). Error bars shown are SEM. (FIG. 6B) A representative image from the western blot analysis is shown. (FIG. 6C) The TPI^{sugarkill} flies overexpressing Zuo1-GFP were compared to a TPI^{sugarkill} homozygote control and were found to have a reduced lifespan by analysis with PRISM software (5 day median lifespan, compared to the 7 day median lifespan observed in TPI^{sugarkill} flies, p<0.05).

FIGS. 7A-7B: E105D TPI is unstable compared to wildtype TPI protein in human fibroblasts. Total TPI protein levels in extracts from wild type control and patient (TPI^{E105D/E105D}) fibroblasts (FB104) were compared by Western blot. The graph shown in FIG. 7A quantifies the Western blot data shown in FIG. 7B. Beta-tubulin was used as a loading control for the Western blot analysis. TPI protein in patient cells was significantly reduced compared to the control when assessed using a Student's t-test (** p<0.01).

FIGS. 8A-8B: Treatment with the Hsp90 inhibitor Luminespib results in an increase in TPI levels in patient TPI^{E105D/E105D} fibroblasts. (FIG. 8A) Densitometry was completed on Western blots and the results were analyzed for significance by Student's t-test (n=4, **P<0.01). Error bars shown are SEM. (FIG. 7B) A representative Western blot was conducted following treatment of patient line FB104 with 200 nM Luminespib for 48 hours at 37° C.

FIGS. 9A-9D: Creation of knockin cell line. (FIGS. 9A-9B) Confocal microscopy of stable human embryonic kidney (HEK) cells expressing the common triose phosphate isomerase deficiency (TPI Df) protein fused with green fluorescent protein (HEK293 GFP-TPI^{E105D}) shows predominantly cytosolic localization of mutant TPI. Images are three-dimensional (3D) reconstructions of a z-series acquired on an Opera Phenix High-Content Screening reader (PerkinElmer) using a 63× water immersion objective. FIG. 9A and FIG. 9B are 3D reconstructions in MetaXpress (Molecular Devices); image in FIG. 9B is a blow-up of selected cells in FIG. 9A. Scale bars: 70 μm and 20 μm, respectively. (FIG. 9C) A 3D rendering in Harmony 5.0 (PerkinElmer) of a z-series of images acquired with a 63× water immersion objective. (FIG. 9D) A representative Western blot of TPI in a sample of the stable Flip-In (Invitrogen) HEK293 cell line. Beta tubulin was used as a loading control. Sizes of molecular weight markers are in kDa.

FIGS. 10A-10E: Luminespib increases GFP-TPI expression that is quantifiable by high content analysis. (FIG. 10A) Pre-screen of seven putative protein stabilizing compounds in HEK293 GFP-TPI^{E105D} identifies luminespib as a strong inducer of GFP-TPI with low cytotoxicity. X-axis shows percent PI positive cells; y-axis is total TPI-GFP intensity per cell; lines represent the mean of vehicle-treated cells. (FIG. 10B) Luminespib treatment results in dose-dependent increases in GFP-TPI with loss of cell attachment but little cell death. Data are mean±SD from five independent biological repeats, each performed in quadruplicate. (FIGS. 10C-10D) Luminespib increases GFP-TPI expression and causes apoptosis in HEK293 cells, but high levels of GFP-TPI do not originate from dying cells. (FIG. 10C) Fluorescence micrographs of TPI-GFP and PI from vehicle-treated and luminespib-treated cells indicate that dead cells do not express GFP-TPI. Luminespib concentration=99 nM. Bar=30 μm. (FIG. 10D) Flow cytometry of non-permeabilized but EDTA-detached cells stained with PI. Luminespib causes apoptosis but apoptotic cells are GFP low, consistent with images in FIG. 10C. Histograms show GFP distribution in the viable cells. Data are from a single well on a 96-well plate; insert shows three technical replicates with mean and SD. Scatterplot is from 30,000 cells treated with DMSO (left) or 99 nM luminespib (right). (FIG. 10E) Miniaturization and preliminary HTS performance in 384-well plates indicates the assay meets universally accepted HTS criteria (Z′>0.5, CV<10%; MIN, DMSO; MAX, 100 nM luminespib, n=144).

FIGS. 11A-11C: Pilot screen of the NIH Clinical Collection. The NIH clinical collection was screened in HEK293 TPI-GFP^{E105D} cells at two concentrations and at two different time points. (FIG. 11A) Scatter plot showing total TPI-GFP intensity per cell, normalized to vehicle control, versus cell loss at the 10 μM, 48 hour condition. Data points represent the averages of duplicate plates. Horizontal and vertical lines represent mean+3 SD of TPI-GFP expression in DMSO treated cells and 50% reduction in cell density, respectively. Marked compounds were chosen based on those cutoffs. (FIG. 11B) Heatmap comparing activity of hits against the same concentration at 24 hours (column 2), and a lower concentration (1 μM) for both time points (columns 3 and 4). Compounds that were active at the 10 μM, 48 hour condition are shown. (FIG. 11C) Comprehensive heatmap of all positive compounds that were active at any condition.

FIGS. 12A-12C: Hit confirmation in TPI-Df patient fibroblasts. (FIG. 12A) Ten-point, twofold dose-response in HEK293 TPI-GFP^{E105D} showing differences in EC₅₀ and magnitude of response for two novel inducers of mutant TPI identified from the NIH Clinical Collection (itavastatin and resveratrol) and selected structural analogs. Table shows EC₅₀ values in μM as mean±standard deviation (SD) from (n) independent biological repeats. Data in graphs are the mean±standard error of the mean (SEM) of quadruplicate wells in a 384-well plate. (FIG. 12B) Representative Western blot of mutant TPI protein levels in [E105D/E105D] patient fibroblasts treated with luminespib (200 nM), resveratrol (100 μM), and itavastatin (5 μM). (FIG. 12B, right) Quantification of mutant TPI levels by densitometry. Data show mean±SD of five independent biological replicates (closed circles). t-test with Welch correction p-values shown compared with DMSO vehicle control. (FIG. 12C) Mutant TPI protein levels by Western blot in [Q181P/E104D] patient fibroblasts treated with 10 μM resveratrol or itavastatin. Data are mean±SEM of three biological replicates (* p<0.05; **p<0.005).

FIG. 13: Three-day variability. Two full 384-well microplates were plated and treated on three consecutive days with 1% DMSO (MIN) or 200 nM luminespib using equipment to be used in HTS. SD, standard deviation; CV, coefficient of variance; S/B ratio, signal-to-background ratio. Scatter plots illustrate day-to-day performance; tables show calculated intra-plate and inter-plate variability statistics.

FIGS. 14A-14C: Dose-response confirmation of hits from the NIH Clinical Collection by high-content analysis confirms activity of resveratrol and artesunate in an additional TPI-GFP cell line (U2OS-TPI-E105D) and patient fibroblasts. HEK293 TPI^{E105D-GFP} and U2OS TPI^{E105D-GFP} data are total GFP levels per cell; patient fibroblast data are total fluorescence measurements in cells stained with an anti-TPI antibody followed by a Cy3-conjugate secondary. Data in graphs are the mean±standard deviation of the mean of quadruplicate wells in a 384-well plate.

### SEQUENCE LISTING

The amino acid sequence listed in the accompanying sequence listing is shown using standard three letter code for amino acids, as defined in 37 C.F.R. 1.822. The Sequence Listing is submitted as an ASCII text file, created on Dec. 21, 2021, 2.54 KB, which is incorporated by reference herein. In the accompanying sequence listing:

is the amino acid sequence of human TPI,
deposited under GenBank Accession No. NP_000356.1.
SEQ ID NO: 1
MAPSRKFFVGGNWKMNGRKQSLGELIGTLNAAKVPADTEVVCAPPTAYI
DFARQKLDPKIAVAAQNCYKVTNGAFTGEISPGMIKDCGATWVVLGHSE
RRHVFGESDELIGQKVAHALAEGLGVIACIGEKLDEREAGITEKVVFEQ
TKVIADNVKDWSKVVLAYEPVWAIGTGKTATPQQAQEVHEKLRGWLKSN
VSDAVAQSTRIIYGGSVTGATCKELASQPDVDGFLVGGASLKPEFVDII
NAKQ

### DETAILED DESCRIPTION

Abbreviations
DMSO dimethyl sulfoxide
FSC forward scatter
GFP green fluorescent protein
Hsp heat shock protein
HTS high-throughput screen
PI propidium iodide
PQC protein quality control
RAC ribosome associated chaperone
RNAi RNA interference
S/B signal-to-background
SD standard deviation
SSC side scatter
TPI triosephosphate isomerase
TPI Df triosephosphate isomerase deficiency
UPS ubiquitin-proteasome system
UV ultraviolet

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various embodiments, the following explanations of terms are provided:

ACO1 (Aconitase 1): A human gene encoding a bifunctional, cytosolic protein that functions as an essential enzyme in the tricarboxylic acid (TCA) cycle and interacts with mRNA to control the levels of iron inside cells. When cellular iron levels are high, the ACO1 protein binds to a 4Fe-4S cluster and functions as an aconitase, which are iron-sulfur proteins that function to catalyze the conversion of citrate to isocitrate. When cellular iron levels are low, the protein binds to iron-responsive elements (IREs), which are stem-loop structures found in the 5′ UTR of ferritin mRNA, and in the 3′ UTR of transferrin receptor mRNA. When the protein binds to IRE, it results in repression of translation of ferritin mRNA, and inhibition of degradation of the otherwise rapidly degraded transferrin receptor mRNA. The ACO1 protein has been identified as a moonlighting protein based on its ability to perform mechanistically distinct functions. Alternative splicing of this gene results in multiple transcript variants. Nucleotide and protein sequences of ACO1 are publicly available, such as under National Center for Biotechnology Information (NCBI) Gene ID 48.

ACTR3 (Actin related protein 3): A human gene encoding a protein that is a major constituent of the ARP2/3 complex. This complex is located at the cell surface and is essential to cell shape and motility through lamellipodial actin assembly and protrusion. Three transcript variants encoding two different isoforms have been found for this gene. Nucleotide and protein sequences of ACTR3 are publicly available, such as under NCBI Gene ID 10096.

Administration: The introduction of a composition, such as a small molecule therapeutic, into a subject by a chosen route. Administration can be local or systemic. Exemplary routes of administration include, but are not limited to, oral, injection (such as subcutaneous, intramuscular, intradermal, intraperitoneal, intravenous), sublingual, rectal, transdermal (for example, topical), intranasal, vaginal, and inhalation routes.

Agent: Any substance, compound or drug that is useful for achieving a particular outcome. For example, the agent can be a substance, compound or drug capable of modulating protein stability, such as increasing protein stability and/or decreasing protein degradation. In some embodiments, the agent is a compound that promotes stability of a mutant TPI protein. In some embodiments, the agent is a therapeutic agent, such as a therapeutic agent for the treatment of TPI deficiency.

Amino acid substitution: The replacement of an amino acid in a polypeptide with one or more different amino acids. The amino acid substitution can be a conservative amino acid substitution or a non-conservative amino acid substitution. In some examples, the amino acid substitution replaces a glutamic acid with an aspartic acid at position 105 of human TPI (E105D) of SEQ ID NO: 1. Other exemplary amino acid substitutions are listed in Table 1.

ANAPC10 (Anaphase promoting complex subunit 10): A human gene encoding a core subunit of the anaphase-promoting complex (APC), a ubiquitin protein ligase that is essential for progression through the cell cycle. APC initiates sister chromatid separation by ubiquitinating the anaphase inhibitor securin and triggers exit from mitosis by ubiquitinating cyclin B, the activating subunit of cyclin-dependent kinase-1 (CDK1). Nucleotide and protein sequences of ANAPC10 are publicly available, such as under NCBI Gene ID 10393.

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and is functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8:e1002388, 2012; and Kunik et al., Nucleic Acids Res 40(Web Server issue):W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by methods known to those of skill in the art. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one instances, all CDRs are from the donor immunoglobulin in a humanized immunoglobulin. Constant regions need not be present, but if they are, they must be substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Antisense compound: An oligomeric compound that is at least partially complementary to the region of a target nucleic acid molecule to which it hybridizes. As used herein, an antisense compound that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression. In some examples, the target nucleic acid molecule is an ACO1, ACTR3, ANAPC10, ASF1A, CDC34, CPB1, CRAA, CUL3, DNAAF4, DNAJB11, DNAJC2, DNAJC15, ERP29, GTPB6, HSP90AA2, HSPB1, HUWE1, MPV17L2, PSMA7, SEC61 G, TOMM22, UBE2B, UBE2Q1, USP14, USP16 or USP45 nucleic acid.

Nonlimiting examples of antisense compounds include primers, probes, antisense oligonucleotides, siRNAs, miRNAs, shRNAs and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound.

Antisense oligonucleotide: A single-stranded antisense compound that is a nucleic acid-based oligomer. An antisense oligonucleotide can include one or more chemical modifications to the sugar, base, and/or internucleoside linkages. Generally, antisense oligonucleotides are “DNA-like” such that when the antisense oligonucleotide hybridizes to a target mRNA, the duplex is recognized by RNase H (an enzyme that recognizes DNA:RNA duplexes), resulting in cleavage of the mRNA.

Artesunate (4-oxo-4-[[(1R,4S,5R,8S,9R,10S,12R,13R)-1,5,9-trimethyl-11,14,15,16-tetraoxatetracyclo[10.3.1.0^{4,13}.0^{8,13}]hexadecan-10-yl]oxy]butanoic acid): A water-soluble, semi-synthetic derivative of the sesquiterpine lactone artemisinin with anti-malarial, anti-schistosomiasis, antiviral, and potential anti-neoplastic activities. The chemical formula of artesunate is C₁₉H₂₈O₈.

ASF1A (anti-silencing function 1A histone chaperone): A human gene encoding a member of the H3/H4 family of histone chaperone proteins and is similar to the anti-silencing function-1 gene in yeast. The ASF1A protein is a key component of a histone donor complex that functions in nucleosome assembly. It interacts with histones H3 and H4, and functions together with a chromatin assembly factor during DNA replication and repair. Nucleotide and protein sequences of ASF1A are publicly available, such as under NCBI Gene ID 25842.

CDC34 (Cell division cycle 34, ubiquitin conjugating enzyme): A human gene encoding a member of the ubiquitin-conjugating enzyme family Ubiquitin-conjugating enzymes catalyze the covalent attachment of ubiquitin to other proteins. The CDC34 protein is a part of the large multiprotein complex, which is required for ubiquitin-mediated degradation of cell cycle G1 regulators, and for the initiation of DNA replication. Nucleotide and protein sequences of CDC34 are publicly available, such as under NCBI Gene ID 997.

Cell culture vessel: Any container or solid support suitable for culturing a stable cell line in vitro. Cell culture vessels include, but are not limited to, multi-well plates (e.g., 6-well, 12-well, 24-well, 48-well, 96-well, 384-well or 1536-well plates), cell culture flasks (e.g., T-25, T-75, T-150, T-175 or T-225 flasks), and cell culture dishes (e.g., 35 mm, 60 mm, 100 mm or 150 mm dishes). In some embodiments, the cell culture vessel is comprised of polystyrene or another type of plastic. In some examples, the vessel surface is coated with collagen, serum, laminin, gelatin, poly-L lysine, or fibronectin.

Collagen: The primary protein of the extracellular matrix found in connective tissues. Collagen is often used as a coating on cell culture vessels to promote cell adhesion and/or proliferation.

Contacting: Placement in direct physical association; includes both in solid and liquid form. “Contacting” is often used interchangeably with “exposed.” For example, cultured cells expressing mutant TPI can be contacted with a candidate agent by adding the agent to the cell culture media. As another example, cells cultured in vitro can be contacted with an agent that promotes stability of mutant TPI by adding the agent to the cell culture media. In the context of an in vivo method, “contacting” cells with an agent that promotes stability of mutant TPI refers to administering the agent to a subject, such as a subject with TPI Df.

Control: A reference standard, for example a positive control or negative control. A positive control is known to provide a positive test result. A negative control is known to provide a negative test result. However, the reference standard can be a theoretical or computed result, for example a result obtained in a population. In some examples of the disclosed methods, cells stably expressing TPI^{E105D-GFP} cultured in the absence of a candidate agent serve as the negative control. In some examples, cells stably expressing TPI^{E105D-GFP} cultured in the presence of luminespib are used as the positive control.

CPB1 (Carboxypeptidase B1): A human gene encoding carboxypeptidase B1, a highly tissue-specific protein that is a useful serum marker for acute pancreatitis and dysfunction of pancreatic transplants. Nucleotide and protein sequences of CPB1 are publicly available, such as under NCBI Gene ID 1360.

CRYAA (Crystallin alpha A): A human gene encoding crystallin alpha-A. Mammalian lens crystallins are divided into alpha, beta, and gamma families. Alpha crystallins are composed of two gene products: alpha-A and alpha-B, for acidic and basic, respectively. Alpha crystallins can be induced by heat shock and are members of the small heat shock protein (HSP20) family. They act as molecular chaperones although they do not renature proteins and release them in the fashion of a true chaperone; instead they hold them in large soluble aggregates. Post-translational modifications decrease the ability to chaperone. These heterogeneous aggregates consist of 30-40 subunits; the alpha-A and alpha-B subunits have a 3:1 ratio, respectively. Two additional functions of alpha crystallins are an autokinase activity and participation in the intracellular architecture. The CRYAA protein has been identified as a moonlighting protein based on its ability to perform mechanistically distinct functions. Alpha-A and alpha-B gene products are differentially expressed; alpha-A is preferentially restricted to the lens and alpha-B is expressed widely in many tissues and organs. Defects in this gene cause autosomal dominant congenital cataract (ADCC). Nucleotide and protein sequences of CRYAA are publicly available, such as under NCBI Gene ID 1409.

CUL3 (Cullin 3): A human gene encoding a member of the cullin protein family. The encoded protein plays a critical role in the polyubiquitination and subsequent degradation of specific protein substrates as the core component and scaffold protein of an E3 ubiquitin ligase complex. Complexes including the CUL3 protein may also play a role in late endosome maturation. Mutations in this gene are a cause of type 2E pseudohypoaldosteronism. Alternatively spliced transcript variants encoding multiple isoforms have been observed for this gene. Nucleotide and protein sequences of CUL3 are publicly available, such as under NCBI Gene ID 8452.

DNAAF4 (Dynein axonemal assembly factor 4): A human gene encoding a tetratricopeptide repeat domain-containing protein. The DNAAF4 protein interacts with estrogen receptors and the heat shock proteins, Hsp70 and Hsp90. A homologous protein in rat has a function in neuronal migration in the developing neocortex. A chromosomal translocation involving this gene is associated with a susceptibility to developmental dyslexia. Mutations in this gene are associated with deficits in reading and spelling. Alternative splicing results in multiple transcript variants. Nucleotide and protein sequences of DNAAF4 are publicly available, such as under NCBI Gene ID 161582.

DNAJB11 (DnaJ heat shock protein family (Hsp40) member B11): A human gene encoding a soluble glycoprotein of the endoplasmic reticulum (ER) lumen that functions as a co-chaperone of binding immunoglobulin protein, a 70 kilodalton heat shock protein chaperone required for the proper folding and assembly of proteins in the ER. The DNAJB11 protein contains a highly conserved J domain of about 70 amino acids with a characteristic His-Pro-Asp (HPD) motif and may regulate the activity of binding immunoglobulin protein by stimulating ATPase activity. Nucleotide and protein sequences of DNAJB11 are publicly available, such as under NCBI Gene ID 51726.

DNAJC2 (DnaJ heat shock protein family (Hsp40) member C2): A human gene encoding a member of the M-phase phosphoprotein (MPP) family. This gene encodes a phosphoprotein with a J domain and a Myb DNA-binding domain which localizes to both the nucleus and the cytosol. The DNAJC2 protein is capable of forming a heterodimeric complex that associates with ribosomes, acting as a molecular chaperone for nascent polypeptide chains as they exit the ribosome. This protein was identified as a leukemia-associated antigen and expression of the gene is upregulated in leukemic blasts. Also, chromosomal aberrations involving this gene are associated with primary head and neck squamous cell tumors. Alternatively spliced variants which encode different protein isoforms have been described. Nucleotide and protein sequences of DNAJC2 are publicly available, such as under NCBI Gene ID 27000.

DNAJC15 (DnaJ heat shock protein family (Hsp40) member C15): A human gene encoding a negative regulator of the mitochondrial respiratory chain. The DNAJC15 protein prevents the mitochondria) hyperpolarization state and restricts mitochondria) generation of ATP. The protein also acts as an import component of the TIM23 transiocase complex and stimulates the ATPase activity of HSPA9. Nucleotide and protein sequences of DNAJC15 are publicly available, such as under NCBI Gene ID 29103.

ERP29 (Endoplasmic reticulum protein 29): A human gene encoding a protein that localizes to the lumen of the endoplasmic reticulum (ER). The ERP29 protein is a member of the protein disulfide isomerase (PDI) protein family but lacks an active thioredoxin motif, suggesting that it does not function as a disulfide isomerase. The canonical protein dimerizes and is thought to play a role in the processing of secretory proteins within the ER. Alternative splicing results in multiple transcript variants encoding different isoforms. Nucleotide and protein sequences of ERP29 are publicly available, such as under NCBI Gene ID 10961.

Fluorescence intensity: A measure of photon (light) emission from a fluorophore. In the context of the methods disclosed herein, fluorescence intensity directly correlates with the amount of fluorescent protein present in cells stably expressing the fluorescent protein as a fusion with mutant TPI (such as TPI^{E105D})

Fluorescent protein: A protein that emits light of a certain wavelength when exposed to a particular wavelength of light. Fluorescent proteins include, but are not limited to, green fluorescent proteins (such as GFP, EGFP, AcGFP1, Emerald, Superfolder GFP, Azami Green, mWasabi, TagGFP, TurboGFP and ZsGreen), blue fluorescent proteins (such as EBFP, EBFP2, Sapphire, T-Sapphire, Azurite and mTagBFP), cyan fluorescent proteins (such as ECFP, mECFP, Cerulean, CyPet, AmCyanl, Midori-Ishi Cyan, mTurquoise and mTFP1), yellow fluorescent proteins (EYFP, Topaz, Venus, mCitrine, YPet, TagYFP, PhiYFP, ZsYellow1 and mBanana), orange fluorescent proteins (Kusabira Orange, Kusabira Orange2, mOrange, mOrange2 and mTangerine), red fluorescent proteins (mRuby, mApple, mStrawberry, AsRed2, mRFP1, JRed, mCherry, HcRedl, mRaspberry, dKeima-Tandem, HcRed-Tandem, mPlum, AQ143, tdTomato and E2-Crimson), orange/red fluorescence proteins (dTomato, dTomato-Tandem, TagRFP, TagRFP-T, DsRed, DsRed2, DsRed-Express (T1) and DsRed-Monomer) and modified versions thereof.

Fusion protein: A protein containing amino acid sequence from at least two different (heterologous) proteins or peptides. Fusion proteins can be generated, for example, by expression of a nucleic acid sequence engineered from nucleic acid sequences encoding at least a portion of two different (heterologous) proteins. To create a fusion protein, the nucleic acid sequences must be in the same reading frame and contain no internal stop codons. Fusion proteins, particularly short fusion proteins, can also be generated by chemical synthesis. In the context of the present disclosure, GFP and TPI^{E105D} are two heterologous proteins joined to form a fusion protein. However, GFP can be replaced with any fluorescent protein.

GTPBP6 (GTP binding protein 6): A human gene encoding a GTP binding protein that is located in the pseudoautosomal region (PAR) at the end of the short arms of the X and Y chromosomes. Nucleotide and protein sequences of GTPBP6 are publicly available, such as under NCBI Gene ID 8225.

Heterologous: Originating from a different genetic source or species.

HSP9OAA2 (Heat shock protein 90 alpha family class A member 2, pseudogene): A human gene encoding a cytosolic HSP90 protein. HSP90 proteins are highly conserved molecular chaperones that have key roles in signal transduction, protein folding, protein degradation, and morphologic evolution. HSP90 proteins normally associate with other cochaperones and play important roles in folding newly synthesized proteins or stabilizing and refolding denatured proteins after stress. Nucleotide and protein sequences of HSP90AA2 are publicly available, such as under NCBI Gene ID 3324.

HSPB1 (Heat shock protein family B (small) member 1): A human gene encoding a member of the small heat shock protein (HSP20) family of proteins. In response to environmental stress, the HSPB1 protein translocates from the cytoplasm to the nucleus and functions as a molecular chaperone that promotes the correct folding of other proteins. This protein plays an important role in the differentiation of a wide variety of cell types. Expression of HSPB1 is correlated with poor clinical outcome in multiple human cancers, and may promote cancer cell proliferation and metastasis, while protecting cancer cells from apoptosis. Mutations in this gene have been identified in human patients with Charcot-Marie-Tooth disease and distal hereditary motor neuropathy. Nucleotide and protein sequences of HSPB1 are publicly available, such as under NCBI Gene ID 3315.

HUWE1 (HECT, UBA and WWE domain containing E3 ubiquitin protein ligase 1): A human gene encoding a protein containing a C-terminal HECT (E6AP type E3 ubiquitin protein ligase) domain that functions as an E3 ubiquitin ligase. The HUWE1 protein is required for the ubiquitination and subsequent degradation of the anti-apoptotic protein myeloid cell leukemia sequence 1 (Mc11). HUWE1 also ubiquitinates the p53 tumor suppressor, core histones, and DNA polymerase beta. Mutations in the HUWE1 gene are associated with Turner type X-linked syndromic cognitive disability. Nucleotide and protein sequences of HUWE1 are publicly available, such as under NCBI Gene ID 10075.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N Y, 1989, chapters 9 and 11, herein incorporated by reference.

Typically, “stringent conditions” encompass conditions under which hybridization will only occur if there is less than 25% mismatch between the hybridization molecule and the target sequence. “Stringent conditions” may be broken down into particular levels of stringency for more precise definition. Thus, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

Inhibit expression or activity: As used herein, a compound that inhibits expression or activity of a gene selected from ACO1, ACTR3, ANAPC10, ASF1A, CDC34, CPB1, CRAA, CUL3, DNAAF4, DNAJB11, DNAJC2, DNAJC15, ERP29, GTPB6, HSP90AA2, HSPB1, HUWE1, MPV17L2, PSMA7, SEC61G, TOMM22, UBE2B, UBE2Q1, USP14, USP16 and USP45 is a compound that reduces the level of the mRNA or protein encoded by the gene in a cell or tissue, or reduces (including eliminates) one or more activities of the gene product. For example, an antisense compound targeting ACO1 inhibits expression of ACO1 by promoting the degradation of ACO1 mRNA, thereby reducing the level of ACO1 protein. In some embodiments, expression of a gene product (such as an ACO1, ACTR3, ANAPC10, ASF1A, CDC34, CPB1, CRAA, CUL3, DNAAF4, DNAJB11, DNAJC2, DNAJC15, ERP29, GTPB6, HSP90AA2, HSPB1, HUWE1, MPV17L2, PSMA7, SEC61G, TOMM22, UBE2B, UBE2Q1, USP14, USP16 or USP45 gene product) is inhibited at least 10%, at least 25%, at least 50%, at least 75%, at least 90%, or at least 95% relative to a control, such as untreated control cells. As another example, an antibody or small molecule that specifically binds or targets ACO1 may inhibit activity of the ACO1 protein by preventing the ACO1 protein from interacting with another protein or by inhibiting enzymatic activity of the protein. In some embodiments, activity of the gene product (such as an ACO1, ACTR3, ANAPC10, ASF1A, CDC34, CPB1, CRAA, CUL3, DNAAF4, DNAJB11, DNAJC2, DNAJC15, ERP29, GTPB6, HSP90AA2, HSPB1, HUWE1, MPV17L2, PSMA7, SEC61G, TOMM22, UBE2B, UBE2Q1, USP14, USP16 or USP45 gene product) is inhibited at least 10%, at least 25%, at least 50%, at least 75%, at least 90%, or at least 95% relative to an untreated control.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and cells that have been “isolated” include those purified by standard purification methods. Isolated does not require absolute purity, and can include proteins, peptides, nucleic acids, or cells that are at least 50% pure, such as at least 75%, 80%, 90%, 95%, 98%, 99%, or even 99.9% pure.

Itavastatin: A small molecule in the statin family having the molecular formula C₂₅H₂₃CaFNO₄⁺. Itavastatin has been used to lower cholesterol and triglyceride levels. Itavastatin is also known as LIVALO™, pitavastatin, itabavastatin, nisvastatin, NK-104 and NKS-104.

Luminespib: A small molecule inhibitor of heat shock protein 90 (Hsp90). Luminespib, which is also known as NVP-AUY992, has the chemical formula C₂₆H₃₁N₃O₅. MicroRNA (miRNA): Single-stranded RNA molecules that regulate gene expression. miRNAs are generally 21-23 nucleotides in length. miRNAs are processed from primary transcripts known as pri-miRNA to short stem-loop structures called pre-miRNA and finally to functional miRNA. Mature miRNA molecules are partially complementary to one or more messenger RNA molecules, and their primary function is to down-regulate gene expression. MicroRNAs regulate gene expression through the RNAi pathway.

Missense mutation: A single nucleotide change in a nucleic acid sequence resulting in a codon that codes for a different amino acid.

MPV17L2 (MPV17 mitochondrial inner membrane protein like 2): A human gene that is a member of the peroxisomal membrane protein PXMP2/4 family. The protein encoded by this gene is required for the assembly and stability of the mitochondria) ribosome and is a positive regulator of mitochondrial protein synthesis. MPV17L2 is also known as MGC12972 and FKSG24. Nucleotide and protein sequences of PSMA7 are publicly available, such as under NCBI Gene ID 84769.

Oligonucleotide: A plurality of nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compounds, molecules or agents (e.g., an agent that promotes stability of mutant TPI protein). In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition (such as TPI Df) after it has begun to develop. “Ameliorating” refers to the reduction in the number or severity of signs or symptoms of a disease.

PSMA7 (Proteasome 20S subunit alpha 7): A human gene encoding a member of the peptidase T1A family that functions as a 20S core alpha subunit of the 26S proteasome complex. The 26S proteasome is a multicatalytic proteinase complex with a highly ordered structure composed of 2 complexes, a 20S core and a 19S regulator. The 20S core is composed of 4 rings of 28 non-identical subunits; 2 rings are composed of 7 alpha subunits and 2 rings are composed of 7 beta subunits. Proteasomes are distributed throughout eukaryotic cells at a high concentration and cleave peptides in an ATP/ubiquitin-dependent process in a non-lysosomal pathway. The PSMA7 protein interacts with the hepatitis B virus X protein and plays a role in regulating hepatitis C virus internal ribosome entry site (IRES) activity, which is essential for viral replication. The encoded protein also plays a role in the cellular stress response by regulating hypoxia-inducible factor-1alpha. Nucleotide and protein sequences of PSMA7 are publicly available, such as under NCBI Gene ID 5688.

Resveratrol (3,5,4′-trihydroxy-trans-stilbene): A natural polyphenol compound produced by many types of plant species and which is found in red wine and some human foods. Resveratrol is believed to have antioxidant and anti-inflammatory properties. The chemical formula of resveratrol is C₁₄H₁₂O₃.

Ribozyme: A catalytic RNA molecule. In some cases, ribozymes can bind to specific sites on other RNA molecules and catalyze the hydrolysis of phosphodiester bonds in the RNA molecules.

RNA interference (RNAi): Refers to a cellular process that inhibits expression of genes, including cellular and viral genes. RNAi is a form of antisense-mediated gene silencing involving the introduction of double stranded RNA-like oligonucleotides leading to the sequence-specific reduction of RNA transcripts. RNA molecules that inhibit gene expression through the RNAi pathway include siRNAs, miRNAs, and shRNAs.

SEC61G (SEC61 translocon subunit gamma): The Sec61 complex is the central component of the protein translocation apparatus of the endoplasmic reticulum (ER) membrane. Oligomers of the Sec61 complex form a transmembrane channel where proteins are translocated across and integrated into the ER membrane. This complex consists of three membrane proteins-alpha, beta, and gamma. This human gene encodes the gamma-subunit protein. Alternatively spliced transcript variants encoding the same protein have been identified. Nucleotide and protein sequences of SEC61G are publicly available, such as under NCBI Gene ID 23480.

Short hairpin RNA (shRNA): A sequence of RNA that makes a tight hairpin turn and can be used to silence gene expression via the RNAi pathway. The shRNA hairpin structure is cleaved by the cellular machinery into siRNA.

Small interfering RNA (siRNA): A double-stranded nucleic acid molecule that modulates gene expression through the RNAi pathway. siRNA molecules are generally 20-25 nucleotides in length with 2-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.”

Small molecule inhibitor: A molecule, typically with a molecular weight less than about 1000 Daltons, or in some embodiments, less than about 500 Daltons, wherein the molecule is capable of modulating, to some measurable extent, an activity of a target molecule (such as stability of a mutant TPI protein).

Stability (of a protein): In the context of the present disclosure, the stability of a protein refers to the rate of turnover (e.g., degradation) of the protein in a cell. The half-life of a protein is directly correlated with stability of the protein—the greater the half-life of a protein the greater the stability of the protein. Stability of a protein can be effected by several factors, including mutations in the protein and external factors, such as the presence of proteases or elevated temperatures. Thus, an agent that “promotes stability” is an agent that inhibits degradation or the rate of degradation of a protein. In some examples, an agent that promotes stability of a protein is an agent that inhibits degradation of a protein by at least 10%, at least 20%, at least 30%, at least 40%, at least 50%, at least 60%, at least 70%, at least 80%, at least 90%, at least 100% or more compared to degradation of the protein in the absence of the agent. In other examples, an agent that promotes stability of a protein is an agent that increases half-life of the protein by at least 10%, at least 20%, at least 30%, at least 40%, at least 50%, at least 60%, at least 70%, at least 80%, at least 90%, at least 100% or more compared to half-life of the protein in the absence of the agent. In yet other examples, an agent that promotes stability of a protein is an agent leads to an increase in accumulation of the protein in a cell, such as an increase of at least 10%, at least 20%, at least 30%, at least 40%, at least 50%, at least 60%, at least 70%, at least 80%, at least 90%, at least 100% or more compared to accumulation of the protein in the absence of the agent.

Statin: A class of drugs that lower cholesterol levels in the blood used to treat hypercholesterolemia. Statins are also known as HMG-CoA reductase inhibitors. Examples of statins include, but are not limited to, itavastatin (LIVALO™), cerivastatin (BAYCOL™ LIPOBAY™), atorvastatin (LIPITOR™), fluvastatin (LESCOL™), lovastatin (ALTOPREV™) pravastatin (PRAVACHOL™), rosuvastatin (CRESTOR™) and simvastatin (ZOCOR™) Subject: Living multi-cellular vertebrate organisms, a category that includes both human and veterinary subjects, including human and non-human mammals, such as non-human primates.

Therapeutically effective amount: A quantity of a specific substance, such as an agent that promotes stability of mutant TPI, sufficient to achieve a desired effect, such as an increase in quantity of mutant TPI in a cell. For instance, the therapeutically effective amount is the amount necessary to treat TPI Df. In some examples, a therapeutically effective amount is the amount necessary to increase stability of mutant TPI protein at least 10%, at least 20%, at least 30%, at least 40%, at least 50%, at least 60%, at least 70%, at least 80%, at least 90%, at least 100% or more compared to stability of the mutant TPI protein in the absence of the therapeutic agent. In some examples, the therapeutically effective amount is the amount necessary to increase the amount of TPI protein in a cell by at least 10%, at least 20%, at least 30%, at least 40%, at least 50%, at least 60%, at least 70%, at least 80%, at least 90%, at least 100% or more compared to the amount of TPI in a cell in the absence of the therapeutic agent.

TOMM22 (Translocase of outer mitochondrial membrane 22): A human gene encoding an integral membrane protein of the mitochondrial outer membrane. The TOMM22 protein interacts with TOMM20 and TOMM40, and forms a complex with several other proteins to import cytosolic preproteins into mitochondria. Nucleotide and protein sequences of TOMM22 are publicly available, such as under NCBI Gene ID 56993.

Transfecting or transfection: Refers to the process of introducing nucleic acid into a cell or tissue. Transfection can be achieved by any of a number of methods, such as, but not limited to, liposomal-mediated transfection, electroporation and injection.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Triosephosphate isomerase (TPI): A metabolic enzyme that catalyzes the interconversion between dihydroxyacetone phosphate (MAP) and D-glyceraldehyde-3-phosphate (G3P) in glycolysis and gluconeogenesis (Rodriguez-Almazan et al., J Biol Chem 283:23254-23263, 2008). The amino acid sequence of wild-type human TPI is set forth herein as SEQ ID NO: 1.

Triosephosphate isomerase deficiency (TPI DO: A disease caused by mutations in the human gene TPI1, encoding the enzyme TPI. TPI Df is an autosomal recessive multisystem disorder characterized by congenital hemolytic anemia, progressive neuromuscular dysfunction, susceptibility to bacterial infection, and cardiomyopathy. The majority of subjects with TPI Df have a missense mutation at position 105 of the TPI protein, resulting in a glutamic acid to aspartic acid substitution (Arya et al., Hum Mutat 10(4):290-294, 1997). Other known mutations in the human TPI protein include those listed in the table below.

TABLE 1
Known mutations in the human TPI protein

| | | | Numbering |
|---|---|---|---|
| Human | Codon | Described in | based on SEQ |
| mutation^{#} | Change | literature | ID NO: 1 |
| V231M | GTG-ATG | | V232M |
| Q181P | CAG-CCG | | Q181P |
| E104D | GAG-GAC | E104D | E105D |
| R189Q | CGA-TGA | R189Q | R190Q |
| M1K | ATG-AAG | | M1K |
| C42Y | TGT-TAT | C41Y | C42Y |
| A63D | GCT-GAT | | A63D |
| G73A | GGG-GCG | G72A | G73A |
| G123R | GGA-AGA | G122R | G123R |
| E146- | GAG-TAG | E146 | E146-(Term) |
| (Term) | | | |
| V155M | GTG-ATG | V154M | V155M |
| I171V | ATT-GTT | I170V | I171V |
| F241L | TTC-CTC | F240L | F241L |
| F241S | TTC-TCC | F240S | F241S |

^{#}As generally known in the field

UBE2B (Ubiquitin conjugating enzyme E2 B): A human gene encoding a member of the E2 ubiquitin-conjugating enzyme family. The modification of proteins with ubiquitin is an important cellular mechanism for targeting abnormal or short-lived proteins for degradation. Ubiquitination involves at least three classes of enzymes: ubiquitin-activating enzymes, or E1s, ubiquitin-conjugating enzymes, or E2s, and ubiquitin-protein ligases, or E3s. The UBE2B enzyme is required for post-replicative DNA damage repair. Its protein sequence is 100% identical to the mouse, rat, and rabbit homologs, which indicates that this enzyme is highly conserved in eukaryotic evolution. Nucleotide and protein sequences of UBE2B are publicly available, such as under NCBI Gene ID 7320.

UBE2Q1 (Ubiquitin conjugating enzyme E2 Q1): A human gene encoding a member of the E2 ubiquitin-conjugating enzyme family. The UBE2Q1 protein is 98% identical to the mouse counterpart. Nucleotide and protein sequences of UBE2Q1 are publicly available, such as under NCBI Gene ID 55585.

USP14 (Ubiquitin specific peptidase 14): A human gene encoding a member of the ubiquitin-specific processing (UBP) family of proteases that is a deubiquitinating enzyme (DUB) with His and Cys domains. The USP14 protein is located in the cytoplasm and cleaves the ubiquitin moiety from ubiquitin-fused precursors and ubiquitinylated proteins. Mice with a mutation that results in reduced expression of the ortholog of this protein are retarded for growth, develop severe tremors by 2 to 3 weeks of age followed by hindlimb paralysis and death by 6 to 10 weeks of age. Alternate transcriptional splice variants, encoding different isoforms, have been characterized. Nucleotide and protein sequences of USP14 are publicly available, such as under NCBI Gene ID 9097.

USP16 (Ubiquitin specific peptidase 16): A human gene encoding a deubiquitinating enzyme that is phosphorylated at the onset of mitosis and then dephosphorylated at the metaphase/anaphase transition. The USP16 protein can deubiquitinate H2A, one of two major ubiquitinated proteins of chromatin. A mutant form of the protein was shown to block cell division in vitro. Alternate transcriptional splice variants, encoding different isoforms, have been characterized. Nucleotide and protein sequences of USP16 are publicly available, such as under NCBI Gene ID 10600.

USP45 (Ubiquitin specific peptidase 45): A human gene encoding a deubiquitylase that binds ERCC1, the catalytic subunit of the XPF-ERCC1 DNA repair endonuclease. This endonuclease is a critical regulator of DNA repair processes, and the deubiquitylase activity of the USP45 protein is important for maintaining the DNA repair ability of XPF-ERCC1. Nucleotide and protein sequences of USP45 are publicly available, such as under NCBI Gene ID 85015.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

### III. Methods of Treating Triosephosphate Isomerase Deficiency

Triosephosphate isomerase deficiency (TPI Df) is a devastating childhood degenerative disease for which there are currently no treatments. The earliest symptom is usually hemolytic anemia that leads to frequent infections. The disease rapidly progresses to muscle weakness, locomotor impairment, and paralysis, ultimately resulting in irreversible brain damage and a respirator-assisted existence until a premature death, usually within 5 years of diagnosis. Reduced stability of mutant TPI protein, resulting from missense mutations in the TPI1 gene, is believed to underlie disease pathogenesis.

Drosophila with the recessive TPI^{sugarkill} allele exhibit progressive locomotor impairment, neuromuscular impairment and reduced longevity, modeling TPI Df in humans TPI^{sugarkill} produces a functional protein that is degraded by the proteasome. Molecular chaperones, such as Hsp70 and Hsp90, contribute to the regulation of TPI^{sugarkill} degradation. In addition, stabilizing the mutant protein through chaperone modulation results in improved TPI deficiency phenotypes. To identify additional regulators of TPI^{sugarkill} degradation, a genome-wide RNAi screen of all known and predicted components of the ubiquitin-proteasome system (UPS) and PQC pathways, and numerous unknown proteins with domains similar to those commonly found in UPS/PQC components, was performed. Of the 430 proteins screened, 25 proteins were identified that important for TPI stability, including many that were not previously known to be involved in its turnover, as well as novel proteins with no known or associated PQC function for any substrate. The screen independently identified several previously known factors involved in TPI degradation, substantiating these findings and indicating that the screen identified the majority of regulators. Many of the novel PQC modulators are of unknown function and all of the identified proteins have a predicted human ortholog. The human orthologs represent therapeutic targets in the treatment of TPI Df. These regulators of mutant TPI are believed to be much less promiscuous than HSP70/HSP90. Thus, therapeutic agents acting on the identified targets are expected to be much less toxic than drugs targeting HSP70/HSP90.

Described herein are methods of treating TPI Df in a subject. In some embodiments, the method includes administering to the subject a therapeutically effective amount of an agent that inhibits expression or activity of aconitase 1 (ACO1), actin related protein 3 (ACTR3), anaphase promoting complex subunit 10 (ANAPC10), anti-silencing function 1A histone chaperone (ASF1A), cell division cycle 34, ubiquitin conjugating enzyme (CDC34), carboxypeptidase B1 (CPB1), crystallin alpha A (CRYAA), cullin 3 (CUL3), dynein axonemal assembly factor 4 (DNAAF4), DnaJ heat shock protein family (Hsp40) member B11 (DNAJB11), DnaJ heat shock protein family (Hsp40) member C2 (DNAJC2), DnaJ heat shock protein family (Hsp40) member C15 (DNAJC15), endoplasmic reticulum protein 29 (ERP29), GTP binding protein 6 (GTPBP6), heat shock protein 90 alpha family class A member 2 (HSP90AA2), heat shock protein family B (small) member 1 (HSPB1), HECT, UBA and WWE domain containing E3 ubiquitin protein ligase 1 (HUWE1), MPV17 mitochondrial inner membrane protein like 2 (MPV17L2), proteasome 20S subunit alpha 7 (PSMA7), SEC61 translocon subunit gamma (SEC61G), translocase of outer mitochondrial membrane 22 (TOMM22), ubiquitin conjugating enzyme E2 B (UBE2B), ubiquitin conjugating enzyme E2 Q1 (UBE2Q1), ubiquitin specific peptidase 14 (USP14), ubiquitin specific peptidase 16 (USP16), or ubiquitin specific peptidase 45 (USP45).

In some embodiments, the agent inhibits expression of the ACO1, ACTR3, ANAPC10, ASF1A, CDC34, CPB1, CRAA, CUL3, DNAAF4, DNAJB11, DNAJC2, DNATC15, ERP29, GTPB6, HSP90AA2, HSPB1, HUWE1, MPV17L2, PSMA7, SEC61G, TOMM22, UBE2B, UBE2Q1, USP14, USP16 or USP45 gene. In some examples, the agent is an antisense compound targeting an ACO1, ACTR3, ANAPC10, ASF1A, CDC34, CPB1, CRAA, CUL3, DNAAF4, DNATB11, DNAJC2, DNATC15, ERP29, GTPB6, HSP90AA2, HSPB1, HUWE1, MPV17L2, PSMA7, SEC61G, TOMM22, UBE2B, UBE2Q1, USP14, USP16 or USP45 nucleic acid. The antisense compound can be, for example, an antisense oligonucleotide, small interfering RNA (siRNA), microRNA (miRNA), short hairpin RNA (shRNA) or ribozyme. Methods of designing and synthesizing antisense compounds, using known or publicly available sequences, are known. In some cases, the antisense compounds include one or more modifications to improve nuclease resistance or increase binding specificity of the compound. For example, an antisense compound can include a modified base, sugar or internucleoside linkage.

In other embodiments, the agent inhibits activity of a protein encoded by the ACO1, ACTR3, ANAPC10, ASF1A, CDC34, CPB1, CRAA, CUL3, DNAAF4, DNAJB11, DNAJC2, DNAJC15, ERP29, GTPB6, HSP90AA2, HSPB1, HUWE1, MPV17L2, PSMA7, SEC61 G, TOMM22, UBE2B, UBE2Q1, USP14, USP16 or USP45 gene. In some examples, the agent is a small molecule inhibitor. In other examples, the agent is a monoclonal antibody. When the agent is a monoclonal antibody, the antibody can be a humanized antibody, a chimeric antibody (such as an antibody having both human and mouse sequences) or a fully human antibody. In some cases, the antibody is an antigen-binding fragment of an antibody, such as a single-chain antibody (scFv). Methods of generating or isolating monoclonal antibodies are well-known.

In some embodiments, the method further includes selecting a subject who has TPI Df prior to treatment. The subject with TPI Df can have any mutation (such as a missense mutation) coding for a mutant TPI protein that exhibits decreased stability compared to the wild-type human protein (such as any mutation listed in Table 1). Thus, in some examples, the subject expresses a mutant form of the TPI protein comprising a glutamic acid to aspartic acid substitution at position 105 (TPI^{E105D}); a valine to methionine substitution at position 232 (TPI^{V232M}); a glutamine to proline substitution at position 181 (TPI^{Q181P}); an arginine to glutamine mutation at position 190 (TPI^{R190Q}); a methionine to lysine substitution at position 1 (TPI^{MIK}); a cysteine to tyrosine substitution at position 42 (TPI^{C42Y}); an alanine to aspartic acid substitution at position 63 (TPI^{A63D}); a glycine to alanine substitution at position 73 (TPI^{G73A}); a glycine to arginine substitution at position 123 (TPI^{G123R}); a glutamic acid to termination substitution at position 146 (TPI^{E146Term}); a valine to methionine substitution at position 155 (TPI^{V155M}); an isoleucine to valine substitution at position 171 (TPI^{I171V}); a phenylalanine to leucine substitution at position 241 (TPI^{F241L}); or a phenylalanine to serine substitution at position 241 (TPI^{F241S}), wherein amino acid numbering is based on SEQ ID NO: 1. In specific examples, the mutant TPI protein is TPI^{E105D}.

In some embodiments, the therapeutically effective amount of the agent is the amount necessary to increase the amount of the mutant TPI protein in cells of the subject by at least 10%, at least 20%, at least 30%, at least 40%, at least 50%, at least 60%, at least 70%, at least 80%, at least 90%, at least 100% or more compared to the amount of mutant TPI in cells of the subject in the absence of the therapeutic agent (such as prior to treatment).

In some embodiments, the agent is administered as part of a composition that includes a pharmaceutically acceptable carrier.

### IV. Methods of Promoting Stability of Mutant TPI

Further disclosed herein are methods of promoting stability of a mutant form of the human TPI protein in human cells. In some embodiments, the method includes contacting the cells with an effective amount of an agent that inhibits expression or activity of aconitase 1 (ACO1), actin related protein 3 (ACTR3), anaphase promoting complex subunit 10 (ANAPC10), anti-silencing function 1A histone chaperone (ASF1A), cell division cycle 34, ubiquitin conjugating enzyme (CDC34), carboxypeptidase B1 (CPB1), crystallin alpha A (CRYAA), cullin 3 (CUL3), dynein axonemal assembly factor 4 (DNAAF4), DnaJ heat shock protein family (Hsp40) member B11 (DNAJB11), DnaJ heat shock protein family (Hsp40) member C2 (DNAJC2), DnaJ heat shock protein family (Hsp40) member C15 (DNAJC15), endoplasmic reticulum protein 29 (ERP29), GTP binding protein 6 (GTPBP6), heat shock protein 90 alpha family class A member 2 (HSP90AA2), heat shock protein family B (small) member 1 (HSPB1), HECT, UBA and WWE domain containing E3 ubiquitin protein ligase 1 (HUWE1), MPV17L2, proteasome 20S subunit alpha 7 (PSMA7), SEC61 translocon subunit gamma (SEC61G), translocase of outer mitochondrial membrane 22 (TOMM22), ubiquitin conjugating enzyme E2 B (UBE2B), ubiquitin conjugating enzyme E2 Q1 (UBE2Q1), ubiquitin specific peptidase 14 (USP14), ubiquitin specific peptidase 16 (USP16), or ubiquitin specific peptidase 45 (USP45).

In some embodiments, the agent inhibits expression of the ACO1, ACTR3, ANAPC10, ASF1A, CDC34, CPB1, CRAA, CUL3, DNAAF4, DNAJB11, DNAJC2, DNAJC15, ERP29, GTPB6, HSP90AA2, HSPB1, HUWE1, MPV17L2, PSMA7, SEC61G, TOMM22, UBE2B, UBE2Q1, USP14, USP16 or USP45 gene. In some examples, the agent is an antisense compound targeting an ACO1, ACTR3, ANAPC10, ASF1A, CDC34, CPB1, CRAA, CUL3, DNAAF4, DNAJB11, DNAJC2, DNAJC15, ERP29, GTPB6, HSP90AA2, HSPB1, HUWE1, MPV17L2, PSMA7, SEC61G, TOMM22, UBE2B, UBE2Q1, USP14, USP16 or USP45 nucleic acid. The antisense compound can be, for example, an antisense oligonucleotide, small interfering RNA (siRNA), microRNA (miRNA), short hairpin RNA (shRNA) or ribozyme. Methods of designing and synthesizing antisense compounds, using known or publicly available sequences, are well-known. In some cases, the antisense compounds include one or more modifications to improve nuclease resistance or increase binding specificity of the compound. For example, an antisense compound can include a modified base, sugar or internucleoside linkage.

In other embodiments, the agent inhibits activity of a protein encoded by the ACO1, ACTR3, ANAPC10, ASF1A, CDC34, CPB1, CRAA, CUL3, DNAAF4, DNAJB11, DNAJC2, DNAJC15, ERP29, GTPB6, HSP90AA2, HSPB1, HUWE1, MPV17L2, PSMA7, SEC61 G, TOMM22, UBE2B, UBE2Q1, USP14, USP16 or USP45 gene. In some examples, the agent is a small molecule inhibitor. In other examples, the agent is a monoclonal antibody. When the agent is a monoclonal antibody, the antibody can be a humanized antibody, a chimeric antibody (such as an antibody having both human and mouse sequences) or a fully human antibody. In some cases, the antibody is an antigen-binding fragment of an antibody, such as a single-chain antibody (scFv). Methods of generating or isolating monoclonal antibodies are well-known.

The mutant TPI can have any mutation (such as an amino acid substitution resulting from a missense mutation in the TPI1 gene) that exhibits decreased stability compared to the wild-type human protein (such as any mutation listed in Table 1). In some examples, the mutant TPI protein comprises a glutamic acid to aspartic acid substitution at position 105 (TPI^{E105D}); a valine to methionine substitution at position 232 (TPI^{V232M}); a glutamine to proline substitution at position 181 (TPI^{Q181P}); an arginine to glutamine mutation at position 190 (TPI^{R190Q}); a methionine to lysine substitution at position 1 (TPI^{MIK}); a cysteine to tyrosine substitution at position 42 (TPI^{C42V}); an alanine to aspartic acid substitution at position 63 (TPI^{A63D}); a glycine to alanine substitution at position 73 (TPI^{G73A}); a glycine to arginine substitution at position 123 (TPI^{G123R}); a glutamic acid to termination substitution at position 146 (TPI^{E146Term}); a valine to methionine substitution at position 155 (TPI^{V155M}); an isoleucine to valine substitution at position 171 (TPI^{I171V}); a phenylalanine to leucine substitution at position 241 (TPI^{F241L}); or a phenylalanine to serine substitution at position 241 (TPI^{F241S}), wherein amino acid numbering is based on SEQ ID NO: 1. In specific examples, mutant TPI protein is TPI^{E105D}.

In some embodiments, the method is an in vitro method. In some examples, the effective amount of the agent is the amount necessary to increase the amount of the mutant TPI protein in the cells by at least 10%, at least 20%, at least 30%, at least 40%, at least 50%, at least 60%, at least 70%, at least 80%, at least 90%, at least 100% or more compared to the amount of mutant TPI in cells in the absence of the agent.

In some embodiments, the agent is administered as part of a composition that includes a pharmaceutically acceptable carrier.

In other embodiments, the method is an in vivo method wherein contacting the cells with the agent comprises administering the agent to a subject. In some examples, the subject is a human subject with TPI Df. In some examples, the effective amount of the agent is the amount necessary to increase the amount of the mutant TPI protein in cells of a subject by at least 10%, at least 20%, at least 30%, at least 40%, at least 50%, at least 60%, at least 70%, at least 80%, at least 90%, at least 100% or more compared to the amount of mutant TPI in cells of the subject in the absence of the agent (such as prior to treatment). In some examples, the agent is administered as part of a composition that includes a pharmaceutically acceptable carrier.

### V. Assay for Identifications of Agents to Promote Stability of Mutant TPI

The vast majority of disease-causing mutations are missense mutations that subtly modulate protein function. Changes in the stability of the modified protein, which is controlled by protein quality control (PQC) pathways, is a common mechanism of disease pathogenesis. Small molecule modulators within the secretory pathway have been an intensive area of research for their therapeutic potential with numerous diseases. However, the majority of proteins are not expressed by the secretory pathway. Thus, a need exists for small molecule PQC modulators of cytosolic proteins, particularly proteins known to be modified in disease states, such as the TPI protein. Mutations in the TPI protein are associated with TPI deficiency, a devastating childhood degenerative disease for which there are currently no treatments. The present disclosure solves this problem by providing a high-throughput assay to screen for compounds capable of enhancing the stability of mutant TPI protein. Compounds that increase the stability of the mutant TPI protein (which increases the level of TPI protein) are identified as agents that can be used to treat TPI deficiency. The present disclosure identifies several small molecule drugs that enhance stability of mutant TPI, including resveratrol, itavastatin or luminespib.

Disclosed herein is a high-throughput assay for selecting candidate compounds capable of enhancing the stability of mutant forms of the TPI protein. Promoting the stability of mutant TPI protein leads to an increase in the total level of TPI protein present in a cell, which improves therapeutic outcomes.

Provided is a method of identifying an agent that promotes stability of a mutant form of the human TPI protein. In the context of the present disclosure, an agent that promotes stability of a protein is an agent that inhibits degradation or the rate of degradation of a protein and/or increases half-life of the protein. In some embodiments, the method includes culturing, in a cell culture vessel, cells stably expressing a fusion protein comprising the mutant TPI protein and a fluorescent protein; contacting the cells with a candidate agent; and measuring fluorescence intensity of the cells. An increase in fluorescence intensity of the cells compared to control cells cultured in the absence of the candidate agent, identifies the candidate agent as an agent that promotes stability of the mutant TPI protein.

The mutant TPI protein can be any modified TPI protein (such as a TPI with a missense mutation) that exhibits decreased stability compared to the wild-type human protein. In some embodiments, the mutant TPI protein includes at least one of the following mutations (amino acid position based on human TPI protein set forth as SEQ ID NO: 1): a glutamic acid to aspartic acid substitution at position 105 (TPI^{E105D}); a valine to methionine substitution at position 232 (TPI^{V232M}); a glutamine to proline substitution at position 181 (TPI^{Q181P}); an arginine to glutamine mutation at position 190 (TPI^{R190Q}); a cysteine to tyrosine substitution at position 42 (TPI^{C42V}); an alanine to aspartic acid substitution at position 63 (TPI^{A63D}); a glycine to alanine substitution at position 73 (TPI^{G73A}); a glycine to arginine substitution at position 123 (TPI^{G123R}); a valine to methionine substitution at position 155 (TPI^{V155M}); an isoleucine to valine substitution at position 171 (TPI^{I171V}); a phenylalanine to leucine substitution at position 241 (TPI^{F241L}); or a phenylalanine to serine substitution at position 241 (TPI^{F241S}). In specific non-limiting examples, the mutant TPI protein includes the E105D mutation (TPI^{E105D}).

The cells used in the disclosed assay can be any human cell line capable of being transfected and stably expressing the mutant TPI-fluorescent protein fusion protein. In some embodiments, the cells are HEK293 cells. In other embodiments, the cells are HeLa cells. One of skill in the art is capable of selecting an appropriate cell line for use in the assay, as well an appropriate medium for culturing the cells.

The disclosed assay measures the level of mutant TPI protein present in the cells by detecting fluorescence resulting from the presence of the fusion protein. The fluorescent protein component of the fusion protein can be any fluorescent protein capable of being detecting, such as by using a standard laboratory plate reader. In some examples, the fluorescent protein is a green fluorescent protein (GFP), or a variant thereof, such as enhanced GFP (EGFP), AcGFP1, Emerald, Superfolder GFP, Azami Green, mWasabi, TagGFP, TurboGFP and ZsGreen. In other embodiments, the fluorescent protein is a blue fluorescent protein (e.g., EBFP, EBFP2, Sapphire, T-Sapphire, Azurite and mTagBFP), a cyan fluorescent protein (e.g., ECFP, mECFP, Cerulean, CyPet, AmCyanl, Midori-Ishi Cyan, mTurquoise and mTFP1), a yellow fluorescent protein (e.g., EYFP, Topaz, Venus, mCitrine, YPet, TagYFP, PhiYFP, ZsYellow1 and mBanana), an orange fluorescent protein (e.g., Kusabira Orange, Kusabira Orange2, mOrange, mOrange2 and mTangerine), a red fluorescent protein (e.g., mRuby, mApple, mStrawberry, AsRed2, mRFP1, JRed, mCherry, HcRedl, mRaspberry, dKeima-Tandem, HcRed-Tandem, mPlum, AQ143, tdTomato and E2-Crimson), or an orange/red fluorescence protein (e.g., dTomato, dTomato-Tandem, TagRFP, TagRFP-T, DsRed, DsRed2, DsRed-Express (T1) and DsRed-Monomer), or a variant thereof. In specific non-limiting examples, the fusion protein comprises TPI^{E105D} fused to GFP (TPI^{E105D-GFP}).

The cell culture vessel for the disclosed assay can be any vessel suitable for culturing the selected cell line. For high-throughput methods, a multi-well plate is desirable. Thus, in some embodiments, the cell culture vessel is a multi-well plate, such as a 6-well, 12-well, 24-well, 48-well, 96-well, 384-well or 1536-well plate. In other embodiments, the cell culture vessel is a cell culture flask, such as a T-25, T-75, T-150, T-175 or T-225 flask. In other embodiments, the cell culture vessel is a cell culture dish, such as a 35 mm, 60 mm, 100 mm or 150 mm dish. In some embodiments, the cell culture vessel is comprised of polystyrene or another type of plastic. In some examples, the vessel surface is coated with collagen, serum, laminin, gelatin, poly-L lysine, or fibronectin. In specific non-limiting examples, the cell culture vessel is a multi-well plate coated in collagen.

In the context of the disclosed method, “contacting” the cells with a candidate agent refers to adding the candidate agent to the cell culture media. The cells are cultured in the presence of the candidate agent for a period of time sufficient to detect any differences in protein stability (e.g., the difference in stability of the mutant TPI protein when cells are cultured in the presence of the candidate agent versus in the absence of candidate agent). In some embodiments, contacting the cells with the candidate agent comprises culturing the cells with the candidate agent for at least 6 hours, at least 12 hours, at least 18 hours, at least 24 hours, at least 30 hours, at least 36 hours, at least 42 hours, at least 48 hours, at least 54 hours, at least 60 hours, at least 66 hours or at least 72 hours. In specific examples, the cells are cultured with the candidate agent for 24 to 72 hours, such as 24 to 48 hours, 48 to 72 hours, 36 to 54 hours, 42 to 60 hours or 42 or 54 hours.

In some embodiments, the candidate agent is a small molecule. In other embodiments, the candidate agent is a peptide inhibitor, a monoclonal antibody, or an antisense compound, such as an antisense oligonucleotide, siRNA or miRNA.

In some embodiments, the step of measuring fluorescence intensity includes optically detecting fluorescence. For example, a standard, commercially available plate reader (e.g., a microplate reader) can be used to detect fluorescence intensity.

In some embodiments, the method is a high-throughput method, such as a method that evaluates at least 50, at least 75, at least 100, at least 150, at least 200, at least 250, at least 300 or at last 350 samples simultaneously (such as by using the same multi-well plate). In some examples, the cell culture vessel is a multi-well plate, such as a 96-well, 384-well or 1536-well plate. In some examples, fluorescence intensity is measured optically using a multi-well plate reader.

In some embodiments of the method, the cells are cultured with at least two, at least three, at least four or at least five different concentrations of the candidate agent. In some embodiments, fluorescence intensity is measured over time, such as at two, three, four, five or more different timepoints.

In some examples, the agent increases the amount of mutant TPI protein in the cells by at least 10%, at least 20%, at least 30%, at least 40%, at least 50%, at least 60%, at least 70%, at least 80%, at least 90%, at least 100% or more compared to the amount of mutant TPI in cells in the absence of the agent.

In specific non-limiting examples, the method for identifying an agent that promotes stability of a mutant form of the human TPI protein includes culturing in a multi-well plate HEK293 cells stably expressing a fusion protein comprising a mutant TPI protein having a glutamic acid to aspartic acid substitution at position 105 (TPI^{E105D}) and a fluorescent protein; contacting the cells with a candidate agent for at least 24 hours; and optically measuring fluorescence intensity of the cells, wherein an increase in fluorescence intensity of the cells compared to control cells cultured in the absence of the candidate agent, identifies the candidate agent as an agent that promotes stability of the mutant TPI protein.

### VI. Use of Agents that Promote TPI Stability to Treat TPI Df

As discussed above, TPI deficiency is a devastating disease with early childhood-onset caused by numerous missense mutations in the TPI1 gene. Studies using patient cells as well as research in Drosophila, suggest that pathogenesis is driven by mutations that destabilize the protein (Torres-Larios, J Biol Chem 283:23254-23263, 2008; De La Mora-De La Mora et al., Biochim Biophys Acta 1834:2702-2711, 2013; Seigle et al., Genetics 179:855-862, 2008; Hrizo and Palladino, Neurobiol Dis 40:676-683, 2010; Roland et al., J Cell Sci 126:3151-3158, 2013; Roland et al., Biochim Biophys Acta Mol Basis Dis 1865:2257-2266, 2019; Orosz et al., IUBMB Life 58:703-715, 2006). Therefore, a need exists to identify therapeutic agents capable of increasing stability (or inhibiting degradation) of mutant TPI. Such agents can be identified using the high-throughput assay disclosed herein (see, for example, section V). Thus, in some embodiments, provided is a method of treating TPI deficiency in a subject by administering to the subject a therapeutically effective amount of an agent identified by the methods disclosed herein.

Further provided is a method of treating a subject who has triosephosphate isomerase deficiency (TPI Df), by administering to the subject a therapeutically effective amount of an agent that promotes stability of a mutant form of the human TPI protein. In some examples, the therapeutically effective amount is the amount necessary to increase the amount of the mutant TPI protein in cells of the subject by at least 10%, at least 20%, at least 30%, at least 40%, at least 50%, at least 60%, at least 70%, at least 80%, at least 90%, at least 100% or more compared to the amount of mutant TPI in cells of the subject in the absence of the therapeutic agent (such as prior to treatment).

The mutant TPI protein is any modified TPI protein (such as TPI resulting from a missense mutation in the TPI gene) that exhibits decreased stability compared to the wild-type human protein. In some embodiments, the mutant TPI protein comprises at least one of the following mutations (amino acid position based on human TPI protein set forth as SEQ ID NO: 1): a glutamic acid to aspartic acid substitution at position 105 (TPI^{E105D)}; a valine to methionine substitution at position 232 (TPI^{V232M}); a glutamine to proline substitution at position 181 (TPI^{Q181P}); an arginine to glutamine mutation at position 190 (TPI^{R190Q}); a cysteine to tyrosine substitution at position 42 (TPI^{2Y}); an alanine to aspartic acid substitution at position 63 (TPI^{A63D}); a glycine to alanine substitution at position 73 (TPI^{G73A)}; a glycine to arginine substitution at position 123 (TPI^{G123R}); a valine to methionine substitution at position 155 (TPI^{V155M}); an isoleucine to valine substitution at position 171 (TPI^{I171V}); a phenylalanine to leucine substitution at position 241 (TPI^{F241L}); or a phenylalanine to serine substitution at position 241 (TPI^{E241S}) In some examples, the subject expresses one of the mutant TPI proteins resulting from the listed mutations. In other examples, the subject expresses two of the mutant TPI proteins resulting from the listed mutations. In specific non-limiting examples, the mutant TPI protein includes the E105D mutation (TPI^{E105D}).

In some embodiments, the agent is a small molecule. In some examples, the agent is luminespib, cerivastatin, itavastatin, ethylestrenol, ethynylestradiol, methyltestosterone, flubendazole, itraconazole, artesunate, resveratrol, isoquercitrin, carvedilol, milrinone, nisoldipine, tadalafil, alprazolam, diphenylcyclopropenone, tegaserod maleate, benproperine phosphate, nelfinavir mesylate, and/or sertraline. In particular examples, the agent is resveratrol, itavastatin, artesunate and/or luminespib.

In some embodiments, the agent is in the statin family. In some examples, the statin is itavastatin and/or cerivastain.

In some embodiments, the agent is a hormone. In some examples, the hormone is ethylestrenol, ethynylestradiol and/or methyltestosterone.

In some embodiments, the agent is an anti-fungal. In some examples, the anti-fungal is flubendazole and/or itraconazole.

In some embodiments, the agent is a natural product. In some examples, the natural product is artesunate, resveratrol and/or isoquercitrin.

In some embodiments, the agent is a vasoactive agent. In some examples, the vasoactive agent is carvedilol, milrinone, nisoldipine and/or tadalafil.

### VII. Exemplary Embodiments

Embodiment 1. A method for identifying an agent that promotes stability of a mutant form of the human triosephosphate isomerase (TPI) protein, comprising:
culturing in a cell culture vessel cells stably expressing a fusion protein comprising the mutant TPI protein and a fluorescent protein;
contacting the cells with a candidate agent; and
measuring fluorescence intensity of the cells, wherein an increase in fluorescence intensity of the cells compared to control cells cultured in the absence of the candidate agent, identifies the candidate agent as an agent that promotes stability of the mutant TPI protein.

Embodiment 2. The method of embodiment 1, wherein the mutant TPI protein comprises:
a glutamic acid to aspartic acid substitution at position 105 (TPI^{E105D});
a valine to methionine substitution at position 232 (TPI^{V232M});
a glutamine to proline substitution at position 181 (TPI^{Q181P});
an arginine to glutamine mutation at position 190 (TPI^{R190Q});
a cysteine to tyrosine substitution at position 42 (TPI^{C42Y});
an alanine to aspartic acid substitution at position 63 (TPI^{A63D});
a glycine to alanine substitution at position 73 (TPI^{G73A});
a glycine to arginine substitution at position 123 (TPI^{G123R});
a valine to methionine substitution at position 155 (TPI^{V155M});
an isoleucine to valine substitution at position 171 (TPI^{I171V});
a phenylalanine to leucine substitution at position 241 (TPI^{E241L}); or
a phenylalanine to serine substitution at position 241 (TPI^{F241S}),
wherein amino acid numbering is based on SEQ ID NO: 1.

Embodiment 3. The method of embodiment 2, wherein the mutant TPI protein is TPI^{E105D}.

Embodiment 4. The method of any one of embodiments 1-3, wherein the cells are HEK293 cells.

Embodiment 5. The method of any one of embodiments 1-4, wherein the fluorescent protein is green fluorescent protein (GFP) or a variant thereof.

Embodiment 6. The method of any one of embodiments 1-5, wherein the cell culture vessel is a multi-well plate.

Embodiment 7. The method of any one of embodiments 1-6, wherein the cell culture vessel is coated in collagen.

Embodiment 8. The method of any one of embodiments 1-7, wherein contacting the cells with the candidate agent comprises culturing the cells with the candidate agent for at least 12 hours, at least 24 hours, at least 36 hours or at least 48 hours.

Embodiment 9. The method of any one of embodiments 1-8, wherein the candidate agent is a small molecule.

Embodiment 10. The method of any one of embodiments 1-9, wherein measuring fluorescence intensity comprises optical detection.

Embodiment 11. The method of any one of embodiments 1-10, wherein the method is a high-throughput method, wherein the cell culture vessel is a multi-well plate and fluorescence intensity is measured optically using a multi-well plate reader.

Embodiment 12. The method of embodiment 11, wherein the cells are cultured with at least two different concentrations of the candidate agent and fluorescence intensity is measured at two or more timepoints.

Embodiment 13. A method of treating a subject who has triosephosphate isomerase deficiency (TPI Df), comprising administering to the subject a therapeutically effective amount of an agent identified by the method of any one of embodiments 1-12.

Embodiment 14. A method of treating a subject who has triosephosphate isomerase deficiency (TPI Df), comprising administering to the subject a therapeutically effective amount of an agent that promotes stability of a mutant form of the human TPI protein.

Embodiment 15. The method of embodiment 14, wherein the mutant TPI protein comprises:
a glutamic acid to aspartic acid substitution at position 105 (TPI^{E105D});
a valine to methionine substitution at position 232 (TPI^{V232M});
a glutamine to proline substitution at position 181 (TPI^{Q181P});
an arginine to glutamine mutation at position 190 (TPI^{R190Q});
a cysteine to tyrosine substitution at position 42 (TPI^{C42V});
an alanine to aspartic acid substitution at position 63 (TPI^{A63D});
a glycine to alanine substitution at position 73 (TPI^{G73A});
a glycine to arginine substitution at position 123 (TPI^{G123R});
a valine to methionine substitution at position 155 (TPI^{V155M});
an isoleucine to valine substitution at position 171 (TPI^{I171V});
a phenylalanine to leucine substitution at position 241 (TPI^{E241L}); or
a phenylalanine to serine substitution at position 241 (TPI^{F241S}).

Embodiment 16. The method of embodiment 15, wherein the mutant TPI protein is TPI^{E105D}.

Embodiment 17. The method of any one of embodiments 14-16, wherein the agent is a small molecule.

Embodiment 18. The method of any one of embodiments 14-17, wherein the agent is luminespib, cerivastain, itavastatin, mevastatin, atorvastatin, ethylestrenol, ethynylestradiol, methyltestosterone, flubendazole, itraconazole, artesunate, resveratrol, isoquercitrin, carvedilol, milrinone, nisoldipine, tadalafil, alprazolam, diphenylcyclopropenone, tegaserod maleate, benproperine phosphate, nelfinavir mesylate, or sertraline.

Embodiment 19. The method of any one of embodiments 14-18, wherein the agent is resveratrol, itavastatin, artesunate, mevastatin or luminespib.

Embodiment 20. A method of treating a subject who has triosephosphate isomerase deficiency (TPI Df), comprising administering to the subject a therapeutically effective amount of resveratrol, itavastatin, artesunate or luminespib.

Embodiment 21. A method of treating triosephosphate isomerase deficiency (TPI Df) in a subject, comprising administering to the subject a therapeutically effective amount of an agent that inhibits expression or activity of aconitase 1 (ACO1), actin related protein 3 (ACTR3), anaphase promoting complex subunit 10 (ANAPC10), anti-silencing function 1A histone chaperone (ASF1A), cell division cycle 34, ubiquitin conjugating enzyme (CDC34), carboxypeptidase B1 (CPB1), crystallin alpha A (CRYAA), cullin 3 (CUL3), dynein axonemal assembly factor 4 (DNAAF4), DnaJ heat shock protein family (Hsp40) member B11 (DNAJB11), DnaJ heat shock protein family (Hsp40) member C2 (DNAJC2), DnaJ heat shock protein family (Hsp40) member C15 (DNAJC15), endoplasmic reticulum protein 29 (ERP29), GTP binding protein 6 (GTPBP6), heat shock protein family B (small) member 1 (HSPB1), HECT, UBA and WWE domain containing E3 ubiquitin protein ligase 1 (HUWE1), MPV17 mitochondrial inner membrane protein like 2 (MPV17L2), proteasome 20S subunit alpha 7 (PSMA7), SEC61 translocon subunit gamma (SEC61G), translocase of outer mitochondrial membrane 22 (TOMM22), ubiquitin conjugating enzyme E2 B (UBE2B), ubiquitin conjugating enzyme E2 Q1 (UBE2Q1), ubiquitin specific peptidase 14 (USP14), ubiquitin specific peptidase 16 (USP16), or ubiquitin specific peptidase 45 (USP45), thereby treating TPI Df in the subject.

Embodiment 22. The method of embodiment 21, wherein the agent inhibits expression of the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 gene.

Embodiment 23. The method of embodiment 22, wherein the agent is an antisense compound targeting an ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 nucleic acid.

Embodiment 24. The method of embodiment 23, wherein the antisense compound comprises an antisense oligonucleotide, small interfering RNA (siRNA), microRNA (miRNA), short hairpin RNA (shRNA) or ribozyme.

Embodiment 25. The method of embodiment 21, wherein the agent inhibits activity of a protein encoded by the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61 G, TOMM22, UBE2B, UBE2Q1, USP14, USP16 or USP45 gene.

Embodiment 26. The method of embodiment 25, wherein the agent is a small molecule inhibitor.

Embodiment 27. The method of embodiment 26, wherein the agent is a monoclonal antibody.

Embodiment 28. The method of any one of embodiments 21-27, further comprising selecting a subject who has TPI Df.

Embodiment 29. The method of any one of embodiments 21-28, wherein the subject expresses a mutant form of the triosephosphate isomerase (TPI) protein, wherein the mutant TPI protein comprises:
a glutamic acid to aspartic acid substitution at position 105 (TPI^{E105D});
a valine to methionine substitution at position 232 (TPI^{V232M});
a glutamine to proline substitution at position 181 (TPI^{Q181P});
an arginine to glutamine mutation at position 190 (TPI^{R190Q});
a cysteine to tyrosine substitution at position 42 (TPI^{C42Y});
an alanine to aspartic acid substitution at position 63 (TPI^{A63D});
a glycine to alanine substitution at position 73 (TPI^{G73A});
a glycine to arginine substitution at position 123 (TPI^{G123R});
a valine to methionine substitution at position 155 (TPI^{V155M});
an isoleucine to valine substitution at position 171 (TPI^{I171V});
a phenylalanine to leucine substitution at position 241 (TPI^{E241L}); or
a phenylalanine to serine substitution at position 241 (TPI^{E241S}),
wherein amino acid numbering is based on SEQ ID NO: 1.

Embodiment 30. The method of embodiment 29, wherein the mutant TPI protein is TPI^{E105D}.

Embodiment 31. A method of promoting stability of a mutant form of the human triosephosphate isomerase (TPI) protein in human cells, comprising contacting the cells with an effective amount of an agent that inhibits expression or activity of aconitase 1 (ACO1), actin related protein 3 (ACTR3), anaphase promoting complex subunit 10 (ANAPC10), anti-silencing function 1A histone chaperone (ASF1A), cell division cycle 34, ubiquitin conjugating enzyme (CDC34), carboxypeptidase B1 (CPB1), crystallin alpha A (CRYAA), cullin 3 (CUL3), dynein axonemal assembly factor 4 (DNAAF4), DnaJ heat shock protein family (Hsp40) member B11 (DNAJB11), DnaJ heat shock protein family (Hsp40) member C2 (DNAJC2), DnaJ heat shock protein family (Hsp40) member C15 (DNAJC15), endoplasmic reticulum protein 29 (ERP29), GTP binding protein 6 (GTPBP6), heat shock protein family B (small) member 1 (HSPB1), HECT, UBA and WWE domain containing E3 ubiquitin protein ligase 1 (HUWE1), MPV17L2, proteasome 20S subunit alpha 7 (PSMA7), SEC61 translocon subunit gamma (SEC61G), translocase of outer mitochondrial membrane 22 (TOMM22), ubiquitin conjugating enzyme E2 B (UBE2B), ubiquitin conjugating enzyme E2 Q1 (UBE2Q1), ubiquitin specific peptidase 14 (USP14), ubiquitin specific peptidase 16 (USP16), or ubiquitin specific peptidase 45 (USP45), thereby promoting stability of the mutant TPI protein.

Embodiment 32. The method of embodiment 31, wherein the agent inhibits expression of the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 gene.

Embodiment 33. The method of embodiment 32, wherein the agent is an antisense compound targeting an ACO1, ACTR3, ANAPC10, ASF1A, CDC34, CPB1, CRAA, CUL3, DNAAF4, DNATB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 nucleic acid.

Embodiment 34. The method of embodiment 33, wherein the antisense compound comprises an antisense oligonucleotide, small interfering RNA (siRNA), microRNA (miRNA), short hairpin RNA (shRNA) or ribozyme.

Embodiment 35. The method of embodiment 31, wherein the agent inhibits activity of a protein encoded by the ACO1, ACTR3, ANAPC10, ASF1A, CDC34, CPB1, CRAA, CUL3, DNAAF4, DNATB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 gene.

Embodiment 36. The method of embodiment 35, wherein the agent is a small molecule inhibitor.

Embodiment 37. The method of embodiment 36, wherein the agent is a monoclonal antibody.

Embodiment 38. The method of any one of embodiments 31-37, wherein the mutant TPI protein comprises:
a glutamic acid to aspartic acid substitution at position 105 (TPI^{E105D});
a valine to methionine substitution at position 232 (TPI^{V232M});
a glutamine to proline substitution at position 181 (TPI^{Q181P});
an arginine to glutamine mutation at position 190 (TPI^{R190Q});
a cysteine to tyrosine substitution at position 42 (TPI^{C42Y});
an alanine to aspartic acid substitution at position 63 (TPI^{A63D});
a glycine to alanine substitution at position 73 (TPI^{G73A});
a glycine to arginine substitution at position 123 (TPI^{G123R});
a valine to methionine substitution at position 155 (TPI^{V155M});
an isoleucine to valine substitution at position 171 (TPI^{I171V});
a phenylalanine to leucine substitution at position 241 (TPI^{E241L}); or
a phenylalanine to serine substitution at position 241 (TPI^{E241S}),
wherein amino acid numbering is based on SEQ ID NO: 1.

Embodiment 39. The method of embodiment 38, wherein the mutant TPI protein is TPI^{E105D}.

Embodiment 40. The method of any one of embodiments 31-39, wherein the method is an in vitro method.

Embodiment 41. The method of any one of embodiments 31-39, wherein the method is an in vivo method wherein contacting the cells with the agent comprises administering the agent to a subject.

Embodiment 42. The method of embodiment 41, wherein the subject is a human subject with triosephosphate isomerase deficiency (TPI Df).

The following examples are provided to illustrate certain particular features and/or embodiments. These examples should not be construed to limit the disclosure to the particular features or embodiments described.

### EXAMPLES

### Example 1: Materials and Methods

This example describes the materials and experimental procedures used for the studies described in Example 2.

### Strain Information

Drosophila melanogaster with the following homozygous genotype: w; DJ694-Gal4; GE-TPI^{sgk} e were generated. The GE-TPI^{sgk} allele (also known as TPI^{sugarkill}) is a genomically engineered allele of TPI that only contains the single point mutation resulting in the M8OT substitution (Roland et al., J Cell Sci 126:3151-3158, 2013). Ebony (e) is linked to the TPI locus and is used as a recessive marker. DJ694-Gal4 is a muscle driver that was used to drive the expression of the RNAi broadly in muscle tissues. Muscle was selected over brain due to the sensitive nature of the nervous system to imbalances in the PQC system and the more reliable effectiveness of RNAi in muscle. Flies were mated with transgenic RNAi strains on the 1st or 2nd chromosome from the Vienna stock collection. Each RNAi line was given an R # to conceal the identity of the factor until after the analyses were completed. Fly strains that utilized p-elements to disrupt gene expression were used for validation studies. Zuo1 overexpressing line was mated with GE-M8OT TPI for examining TPI^{sugarkill} levels in the presence of higher levels of this chaperone. The strains used in the validation studies are listed in Table 2.

TABLE 2
Strains for validation studies

| | | | | Chromo- |
|---|---|---|---|---|
| Description | BL ID | CG# | Gene Name | some |
| p ele disruption | 23866 | CG42616 | Cullin 3 (Predicted: | 2nd |
| | | | ubiquitin-protein | |
| | | | transferase activity) | |
| GFP tagged | 56780 | CG10565 | zuo1 | 2nd |
| p ele disruption | 10483 | CG2720 | HOP | 2nd |

### Analysis of TPI Protein Stability by Western Blot

To induce TPI^{sugarkill} protein instability, 3-5 day old adult flies were incubated at 29° C. for 48 hours (unless otherwise noted). Thoraxes were dissected and three thoraxes per sample were ground with a pestle in 80 μL of 2×SDS-PAGE sample buffer (130 mM Tris (pH 6.8), 4% SDS, 4% beta-mercaptoethanol, 20% glycerol, 0.1% bromophenol blue) with the protease inhibitors: leupeptin (1 μg/μL final concentration), pepstatin A (0.5 μg/μL final concentration), and PMSF (100 μM final concentration). The samples were heated to 96° C. for 3 minutes, and then centrifuged at 5000×g for 1 minute to pellet the debris. Then 20 μL of the samples were loaded and run on a 12% SDS-PAGE gel and transferred to a PVDF (0.45 micron) membrane using a BioRad Semi-Dry Transfer System. Samples were blocked with Odyssey Blocking reagent (Thermofisher Scientific) for 30 minutes at room temperature and then incubated in anti-TPI (1:5000, rabbit polyclonal, FL249, Santa Cruz Biotechnology) and anti-β-tubulin (1:1000, mouse monoclonal E7, Developmental Studies Hybridoma Bank) diluted in Odyssey block overnight at 4° C. Membranes were then washed 6 times in PBST, for 5 minutes each. Following the wash, membranes were incubated 1-3 hours in the dark at room temperature in secondary antibodies: goat anti-mouse-IR-680 (1:20000, A21057, Molecular Probes) and goat anti-mouse-IR-800 (1:20000, NC9209374, Fisher Scientific) in Odyssey blocking reagent with 0.1% Tween 20. Finally, membranes were washed in PBST in the dark for another 6 washes, each lasting 5 minutes. The TPI and β-tubulin bands were visualized on the membrane using the Odyssey Licor System and were quantified using densitometry with Image J software (NIH).

### RNAi Screen Data Analyses

The relative change in protein levels in the RNAi treated samples were compared to a no RNAi control at 25° C. (stable levels of TPI), with an n=4-8 for each RNAi line. The samples were averaged and a Student's t-test was used to compare the relative amount of protein in an RNAi treated sample at 29° C. to the corresponding 29° C. no RNAi Drosophila sample set using Prism. Those samples that had a p-value of less than 0.05 in the first screen were then reevaluated through a second independent set of matings, sample collection and Western blot analyses. The secondary validation screen blots were analyzed for significance with a one-way ANOVA and a Dunnett's post-test. A STRING analysis was conducted on the genes that yielded a p-value of <0.05 in both rounds of screening to examine interactions reported through experimental analysis (online at string-db.org). Human orthologs of the genes were identified by DIOPT v8.0 on Flybase.

### Isolation and Visualization of Poly-Ubiquitinated TPI

Conjugated anti-TPI resin was prepared with TPI antibody (FL249, Santa Cruz Biotechnology) and the Pierce co-immunoprecipitation kit. Homozygous TPI^{sugarkill} flies were fed 1 mg/mL MG132 (Sigma) on 10% sucrose for 24 hours at 25° C. or 29° C. Following incubation, flies were homogenized using a mechanical homogenizer in Pierce co-immunoprecipitation lysis buffer with Protease Inhibitor Mini-Tablets (Pierce), MG132 (0.1 mg/mL, Sigma) and NEM (1 μM, Sigma). Protein concentration was determined by BCA assay (Pierce). The lysates (2 mg) were incubated on a rotator overnight at 4° C. with 100 μl of the conjugated anti-TPI resin. The columns were washed three times with 200 μL of Pierce IP wash buffer with Protease Inhibitor Mini-Tablets (Pierce), MG132 (0.1 mg/mL) and 1 μM NEM. A bead only control provided by the Pierce IP kit served as a negative control. The samples were eluted into 2×SDS-PAGE sample buffer (130 mM Tris (pH 6.8), 4% SDS, 4% beta-mercaptoethanol, 20% glycerol, 0.1% bromophenol blue) with Protease Inhibitor Mini-Tablets (Pierce), MG132 (0.1 mg/mL, Sigma) and NEM (1 μM, Sigma). Western blots were conducted as described above with the exception that the blots were boiled in distilled water for 30 minutes prior to blocking. The antibody used for detection of ubiquitin was anti-ubiquitin (1:1000, 6C1 mouse Santa Cruz Biotechnology). Ubiquitin bands were visualized on the membrane using the Odyssey Licor System.

Validation with Classical Mutant Alleles

Validation of RNAi line results were conducted utilizing available p-element disruption strains and a GFP-tagged Zuo1-overexpressing line (Table 2). Validation included mechanical stress sensitivity analysis and life span analysis. Western blots were conducted as above, but at 25° C. for 5 days for the p-element disruption lines as high heat was lethal for many protein-quality control factor deletion lines. Mechanical stress sensitivity (a.k.a. bang-sensitivity), was performed on day 5 adults (aged at 25° C.) Animals were vortexed and observed for a return to normal behavior following paralysis and seizure, as has been previously described (Seigle et al., Genetics 179:855-862, 2008). The average time to recovery was calculated and the data were examined for significance compared to GE-TPI^{sgk} e homozygote control using a Student's t-test (PRISM). For lifespan analysis, approximately 25 flies aged 1 day were isolated in a vial and were maintained at 25° C. Animals were provided fresh media every 48 hours and deaths were noted. Trials were run in triplicate. Mantel-Cox analysis using PRISM software was used to generate survival curves, determine median lifespan and evaluate significance.

### Culturing Patient Fibroblasts

Patient fibroblasts were obtained from a male TPI deficiency patient via skin punch. The cells were de-identified and are known only as FB104. Fibroblasts were cultured using standard. methods (37° C., 5% CO₂) in complete media (DMEM with 10% serum, 100 U penicillin/100 μg streptomycin/ml (Lanza), 2 mM 1-glutamine (Gibco) and supplemental non-essential amino acids (Gibes)).

### Patient Fibroblast Western Blots

Human fibroblasts were treated with Luminespib (200 TIM, Selleck Chemicals) and D1ISO (0.1%, Sigma Aldrich), then incubated at standard conditions (37° C., 5% CO) for 48 hours. Cells were then trypsinized (0.05% for 5 minutes), pelleted, resuspended in RIPA buffer with protease inhibitors (PMSF (100 μM), leupeptin (1 μg/μL), pepstatin A (0.5 μg/μL)) and were pulse sonicated. Protein concentrations were determined using a BCA assay (Pierce) Immunoblotting was performed on whole protein cell lysates following the addition of an equal volume of 2×SDS-PAGE sample buffer (4% SDS, 4% ii-mercaptoethanol, 130 mM Tris HCl pH 6.5, 20% glycerol). Proteins were resolved by SDS-PAGE (12%) and transferred onto 0.45 μm PVDF membrane. The blots were blocked in Odyssey Blocking Buffer (Licor) and incubated with anti-TPI (1:5000; rabbit polyclonal FL-249; Santa Cruz Biotechnology) or anti-β-tubulin (1:1000; mouse, polyclonal E7-C; Developmental Studies Hybridoma Bank) diluted in Odyssey Blocking Buffer (Licor). Following washes in PBST, the blots were incubated with anti-mouse-IRS00 (Fisher Scientific) and anti-rabbit-18680 (Molecular Probes) both diluted to 1:20,000 in 0.1% Tween 20 blocking buffer. Blots were washed in PBST and developed using Odyssey Infrared Imaging System. Quantification of the scanned images was performed digitally using the Image Studio Ver 5.2 software. Differences in TP1 levels were evaluated for significance by a two-tailed Student's t-test.

### Example 2: Identification of Protein Quality Control Regulators Using a Drosophila Model of TPI Deficiency

This example describes the identification of 25 genes encoding proteins that modulate degradation of mutant TPI protein.

### Primary Genetic Screen

The initial RNAi screen evaluated the expression of 430 different Gal4-dependent UAS-RNAi constructs targeting the expression of a variety of known or predicted protein quality control factors in Drosophila. The initial screening yielded a total of 95 factors that were involved in TPI^{sugarkill} degradation using a Bonferroni correction for multiple comparisons (significant p-value of <0.00011). Of the 430 RNAi lines tested, 217 were reduced with a p<0.05 (without multiple comparison correction). The knock down of these factors generally resulted in increased stabilization of TPI^{sugarkill} compared to corresponding controls with no RNAi, treated at 29° C.

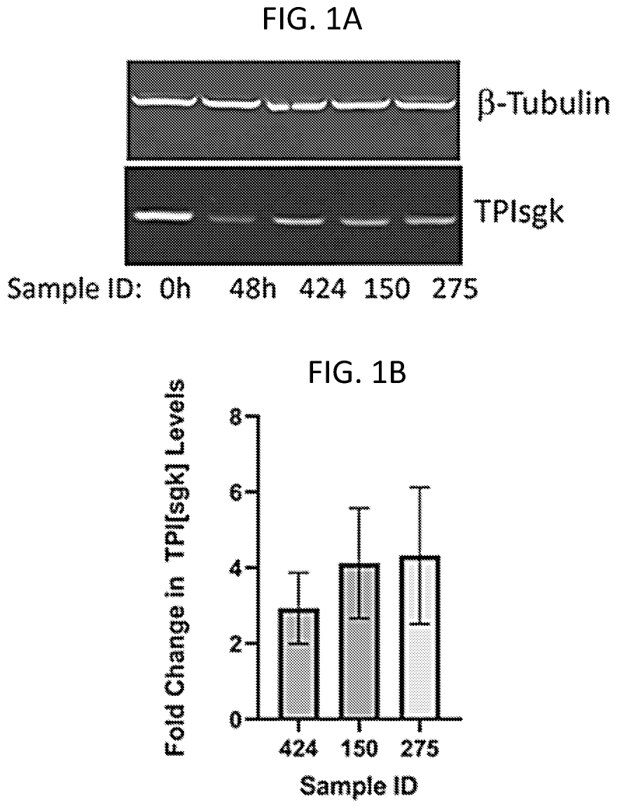

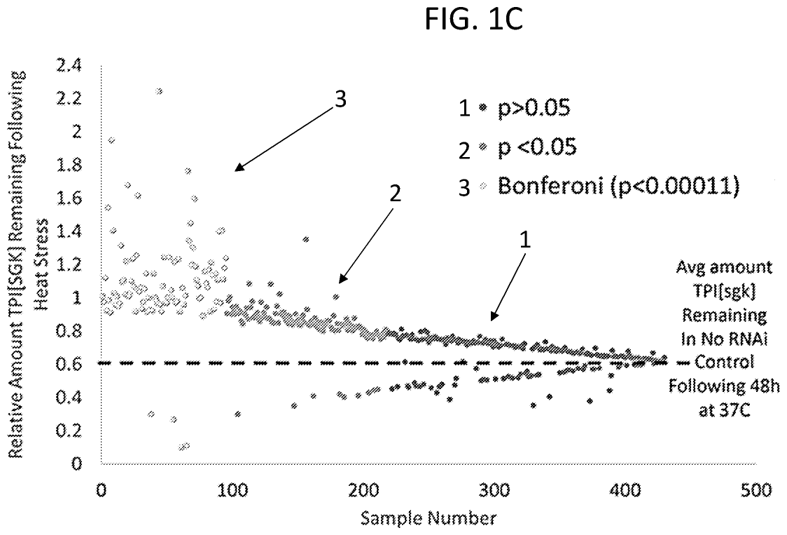

Each RNAi data set was compared to the equivalent 29° C. control. A representative Western blot is shown in FIG. 1A with fold-change in TPI^{sugarkill} levels shown in FIG. 1B. The no RNAi, DJ694 GAL4 driver line expressing TPI^{sugarkill} demonstrated significant degradation of the TPI^{sugarkill} protein following incubation at 29° C. for 48 hours compared to flies of the same genotype incubated at 25° C. for 48 hours. The level of degradation in the control lines was consistent with previously published data that demonstrated ˜60-70% reduction in protein levels (Seigle et al., Genetics 179:855-862, 2008; Hrizo and Palladino, Neurobiol Dis 40:676-683, 2010). The RNAi strains expressing TPI^{sugarkill} were also incubated at 29° C. for 48 hours and the relative amount of TPI protein remaining was compared to the 29° C. no RNAi control on the same blot. This procedure allowed blot variability to be taken into account. Beta-tubulin was used as a loading control for all Western blots as the levels of β-tubulin were robust and stable under the conditions tested.

### Secondary RNAi Screen

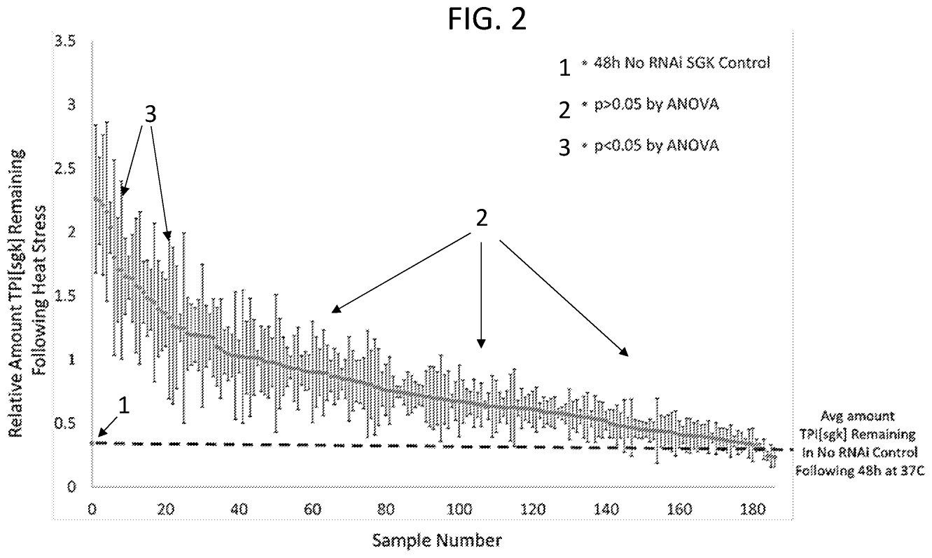

To be inclusive in the second round of screening, the 217 lines identified as potential factors involved in regulating TPI^{sugarkill} degradation (FIG. 1C) were re-evaluated through a second round of crosses and Western blot analyses. This analysis yielded 25 lines that were significantly different compared to the no RNAi treated samples at 29° C. when examined by a One-Way Anova with a Dunnett's Post Test (p<0.05) (FIG. 2 and Table 3). There were several expected hits identified by the screen, such as Hsp70 and Hsp83. Both have been previously shown to regulate TPI^{sugarkill} degradation (Hrizo and Palladino, Neurobiol Dis 40:676-683, 2010).

TABLE 3
Genes identified as potential regulators of TPI degradation

| | | | | Closest |
|---|---|---|---|---|
| | | | | Human |
| | | | | Ortholog |
| | ANOVA | | | via DIOPT |
| R# | p value | CG# | Function | v8.0 |
| 36 | <0.0001 | CG4167 | Heat Shock Gene 67Ba | HSPB1 |
| 62 | 0.0001 | CG1662 | *Proposed Role in Mitochondrial Ribosome | MPV17L2 |
| | | | Assembly | |
| 94 | 0.0262 | CG10565 | *Hsp40 proposed component of RAC, Zuo1 | DNAJC2 |
| 119 | 0.0028 | CG11419 | Anaphase Promoting Complex Subunit 10 | ANAPC10 |
| 161 | <0.0001 | CG2924 | *Proposed E2 ubiquitin-conjugating enzyme | UBE2Q1 |
| 212 | 0.0203 | CG2013 | Ubc6, E2 Ubiquitin Conjugating Enzyme | UBE2B |
| 215 | 0.0257 | CG14981 | Maggie, Mitochondrial Transporting ATPase | TOMM22 |
| 220 | 0.0107 | CG14921 | * Proposed Hsp20 | DNAAF4 |
| 255 | <0.0001 | CG1242 | Heat Shock Protein 83 | HSP90AA2 |
| 307 | <0.0001 | CG7225 | Windbeutel, Toll Signaling in Golgi | ERP29 |
| 314 | <0.0001 | CG8560 | *Proposed metallocarboxypeptidase activity | CPB1 |
| 347 | <0.0001 | CG8184 | HUWE1, E3 Ubiquitin protein ligase | HUWE1 |
| 315 | <0.0001 | CG5116 | *Proposed ribosome binding activity | GTPBP6 |
| 359 | <0.0001 | CG4569 | Proteasome alpha 4 Subunit | PSMA7 |
| 361 | 0.0004 | CG7558 | Arp3 (Actin Related Protein) | ACTR3 |
| 374 | <0.0001 | CG4165 | Ubiquitin Specific Protease 16/45 | USP45 and |
| | | | | USP16 |
| 375 | 0.0004 | CG8860 | *Proposed Translocon Activity | SEC61G |
| 397 | <0.0001 | CG4461 | *Proposed Hsp20 | CRYAA |
| 398 | <0.0001 | CG7656 | *Proposed E2 ubiquitin-conjugating enzyme | CDC34 |
| 399 | <0.0001 | CG42616 | Cullin 3, Ubiquitin Conjugating Enzyme | CUL3 |
| | | | Binding | |
| 412 | <0.0001 | CG5384 | Ubiquitin specific protease 14 | USP14 |
| 421 | 0.0018 | CG32727 | *Proposed Hsp40 | DNAJC15 |
| 435 | 0.0279 | CG6342 | IRP1-B, Aconitase | ACO1 |
| 455 | 0.0035 | CG4164 | Shriveled, Hsp40 | DNAJB11 |
| 504 | 0.0068 | CG9383 | Anti-silencing factor 1 | ASF1A |

*Unknown Function

### STRING Analysis of Regulators

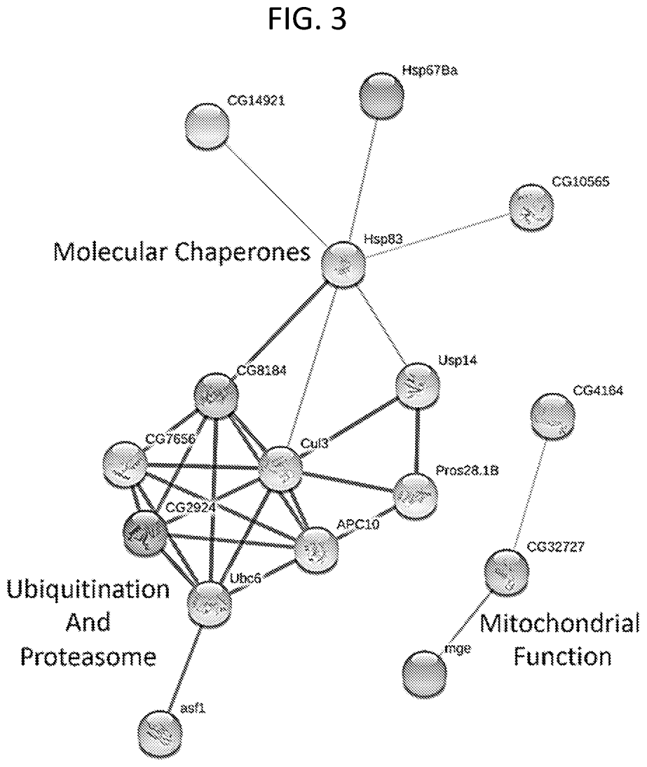

The screening suggested TPI^{sugarkill} degradation is regulated by 10 novel/uncharacterized proteins that are predicted to function in protein quality control and processing. These include putative chaperones, proteases, polyubiquitination enzymes and ribosome associated factors. String analyses use freely available protein-protein interaction data collected from reported experimental evidence in literature and computational prediction methods to generate the node diagram. Thicker lines shown in the STRING analysis indicate multiple levels of support for the reported interactions suggesting that these interactions are highly likely to be valid (FIG. 3). There were three main interconnected nodes identified in the analysis—molecular chaperones, ubiquitination and proteasome function, and a minor unconnected mitochondrial node. As the protein quality control system is highly redundant with many isoforms of the same chaperone and ubiquitination enzyme, it was expected that some classes of chaperones and factors may be enriched if they shared overlapping function. When an interaction map was generated using STRING, the genes identified in the screen appeared to strongly cluster together according to function (FIG. 3). There was a clear node in the interaction map that contains molecular chaperone and factors associated with regulating chaperone function. The chaperone data set was enriched for the Hsp20, Hsp40, Hsp70 and Hsp90 families of proteins. HoP was identified in the initial round of screening but failed to be significant following the more rigorous ANOVA statistical analysis, suggesting it may not have a significant role in TPI^{sugarkill} protein turnover or the RNAi treatment did not robustly knock down this co-chaperone. HiP was not included in the initial screen because a target RNAi line on the 1^{st} or 2^{nd} chromosome was not available when the screen was initiated. Further experiments with classical alleles to see if the canonical HoP, HiP, Hsp70, Hsp90 folding pathway applies to TPI^{sugarkill} may yield additional points of quality control inhibition for therapeutic targeting (Pratt and Dittmar, Trends Endocrinol Metab 9:244-252, 1998; Cox and Johnson, “The role of p23, Hop, immunophilins, and other co-chaperones in regulating Hsp90 function,” In Molecular Chaperones: Springer, pages 45-66, 2011).

In addition to redundancy in the chaperone network, there are often multiple E2 and E3 ubiquitination enzymes that target the same substrate (Stewart et al., Cell Res 26:423-440, 2016; Zheng and Shabek, Annu Rev Biochem 86:129-157, 2017). This redundancy has been observed for some ER PQC ubiquitination proteins, such as Ubc6 and Ubc7 (Rubenstein and Hochstrasser, Cell Cycle 9:4282-4285, 2010). In support of these prior findings, STRING analysis identified a node in the interaction map that is enriched for factors such as E2 ubiquitin conjugating enzymes, E3 ubiquitin ligases, and deubiquitinating enzymes as having a role in TPI^{sugarkill} stability.

Mutant TPI polyubiquitination

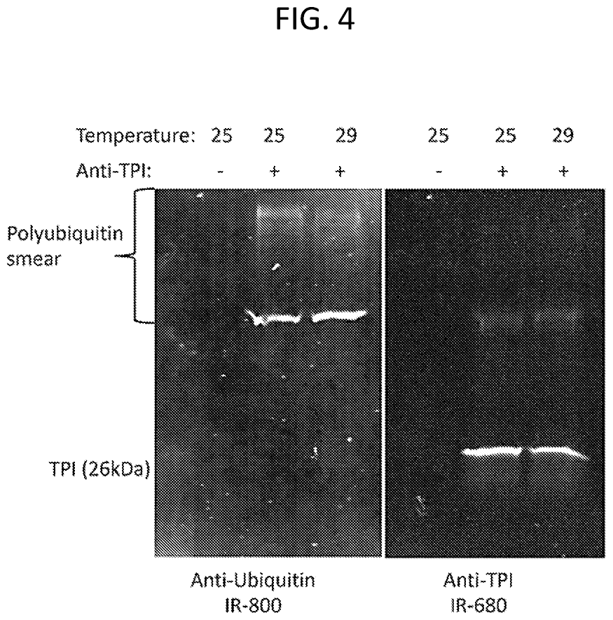

The above findings suggest that TPI^{sugarkill} undergoes polyubiquitination prior to degradation. To test this prediction, an immunoprecipitation and Western blot experiment was conducted to isolate and visualize polyubiquitinated TPI^{sugarkill} As can be seen in FIG. 4, when treated with MG132, isolated TPI^{sugarkill} from fly extract exhibited an accumulation of polyubiquitinated signal. The bead only control did not exhibit any TPI signal or the characteristic high molecular weight incompletely separate products typically associated with polyubiquitination. Polyubiquitinated TPI^{sugarkill} was observed at both 25° C. and 29° C. This was not unexpected as even TPI^{sugarkill} flies incubated at 25° C. exhibit reduced TPI levels compared to flies expressing the wildtype TPI protein (Seigle et al., Genetics 179:855-862, 2008; Hrizo and Palladino, Neurobiol Dis 40:676-683, 2010).

### Validation of Select Mutant TPI Regulators

In order to validate factors identified in the RNAi screen, a select group of target genes were evaluated using available p-element disruptions or overexpression alleles in TPI^{sugarkill} homozygotes (strain information in Table 2). The effect of knock down on gene function by p-element or gene overexpression was evaluated by Western blot, lifespan and mechanical stress sensitivity analyses. P-element disruption is systemic and is expected to knock down/out the targeted protein in all tissues. This had an effect on the overall health of the TPI^{sugarkill} flies, especially at elevated temperatures. Thus, many of the studies with p-element disruptions were conducted at 25° C., where the animals exhibit a modest but significant reduction in protein levels compared to the wildtype control. The investigation of exacerbated proteolysis due to chronic exposure to 29° C. heat stress was not possible due to high death rates in the p-element fly population. Selected for the p-element disruption study were Hsp83 (a known regulator of TPI^{sugarkill} degradation), Cullin 3 (novel putative regulator involved in polyubiquitination identified in the screen) and HiP (a regulator of Hsp90 unable to be tested in the primary screen, but of interest due to the repeated identification of Hsp90 and Hsp70 as factors involved in TPI^{sugarkill} degradation) (Pratt and Dittmar, Trends Endocrinol Metab 9:244-252, 1998; Hrizo and Palladino, Neurobiol Dis 40:676-683, 2010; Cox and Johnson, “The role of p23, Hop, immunophilins, and other co-chaperones in regulating Hsp90 function,” In Molecular Chaperones: Springer, pages 45-66, 2011; Genschik et al., EMBO J 32:2307-2320, 2013).

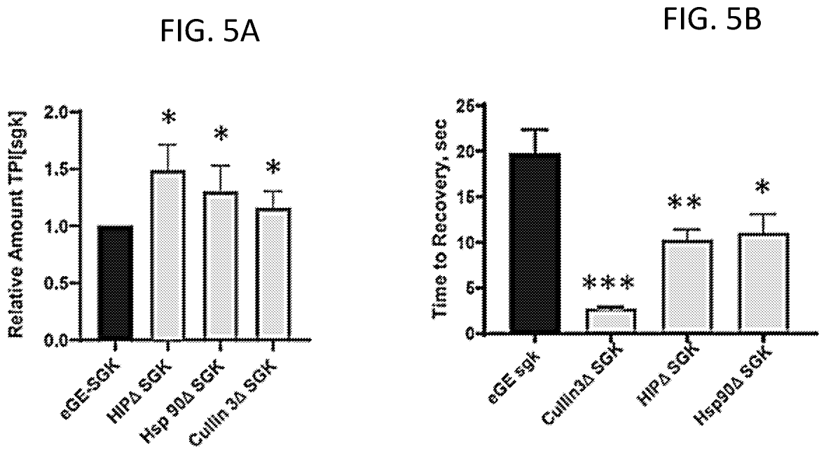

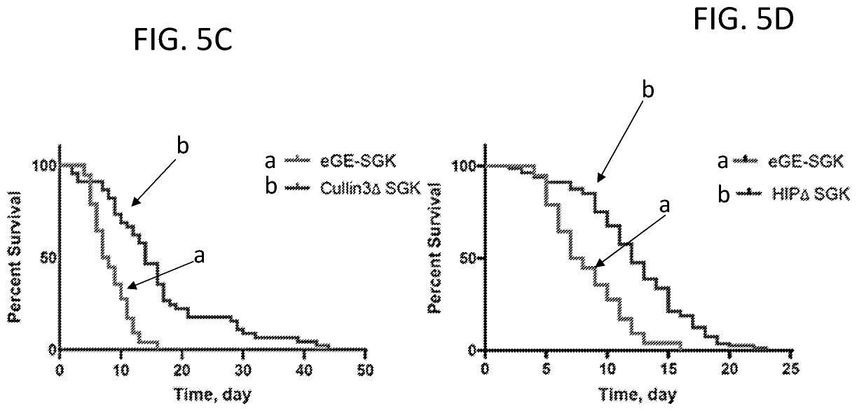

As shown in FIG. 5A, gene disruption alleles of HIP, HSP90 and Cullin 3 all resulted in modest increases in TPI^{sugarkill} protein levels at 25° C. This improvement in protein levels also correlated with an improvement in recovery from paralysis following mechanical stress for all three alleles (FIG. 5B) and increased lifespan compared to the TPI^{sugarkill} homozygote control (FIGS. 5C and 5D). The median lifespan of TPI^{sugarkill} flies at 25° C. is 7.5 days, but the modest increases in protein levels resulting from the p-element disruption was sufficient to increase the median lifespans to 14 days (Cullin 3), 12 days (HiP) and 11 days (Hsp83) with a p value of <0.001 for all analyses. These results indicate that modulating the activity of these target genes results in an improvement in overall function and lifespan in this TPI deficiency model.

### Possible Co-Translational Regulation of Mutant TPI

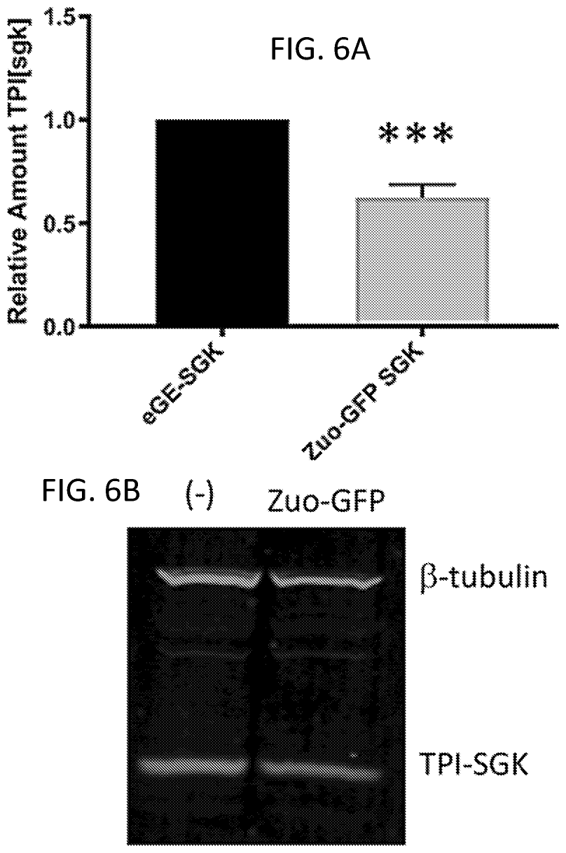

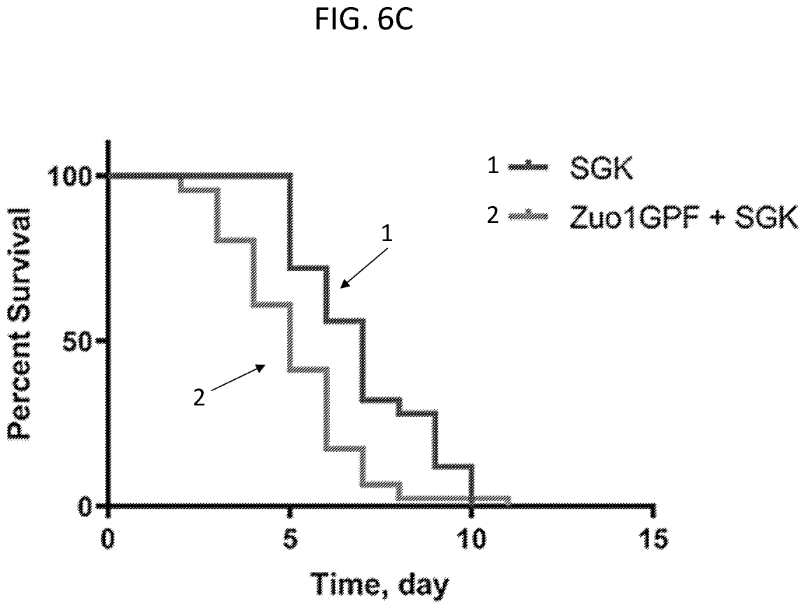

Another set of factors of interest identified by the screen were ribosome associated proteins, including Hsp40, Zuo1/DNAJC2 and CG5116. Zuo1 is found in the Ribosome Associated Chaperone (RAC) complex and assists with the folding of nascent polypeptides as they are synthesized by the ribosome (Amor et al., Prion 9:144-164, 2015; Zhang et al., Nat Struct Mol Biol 24:611-619, 2017). RAC is involved in targeting nascent polypeptides to be degraded co-translationally (Bengtson and Moazeiro, Nature 467:470-473, 2010; Brandman et al., Cell 151:1042-1054, 2012; Defenouillere etal., Proc Natl Acad Sci USA 110:5046-5051, 2013; Lyumkis et al., Proc Natl Acad Sci USA 111:15981-15986, 2014). As the levels of total TPI^{sugarkill} protein continue to destabilize over long periods of time following a shift to 29° C., it is possible that this substrate may be targeted by the RAC for degradation co-translationally (Hrizo and Palladino, Neurobiol Dis 40:676-683, 2010). Due to lethality, a Zuo1 p-element disruption line could not be used for these studies. However, flies with an additional copy of Zuo1 inserted into the chromosome, which results in Zuo1 overexpression, were utilized (Kudron et al., Genetics 208:937-949, 2018). When Zuo1 was overexpressed in TPI^{sugarkill} flies, the stability of the protein was decreased further and corresponded to a worsening of lifespan, suggesting an important role for this chaperone in modulating the protein levels of TPI^{sugarkill} (FIGS. 6A-6C).

### Mutant TPI^{E105D} Protein is Reduced in Patient Fibroblasts

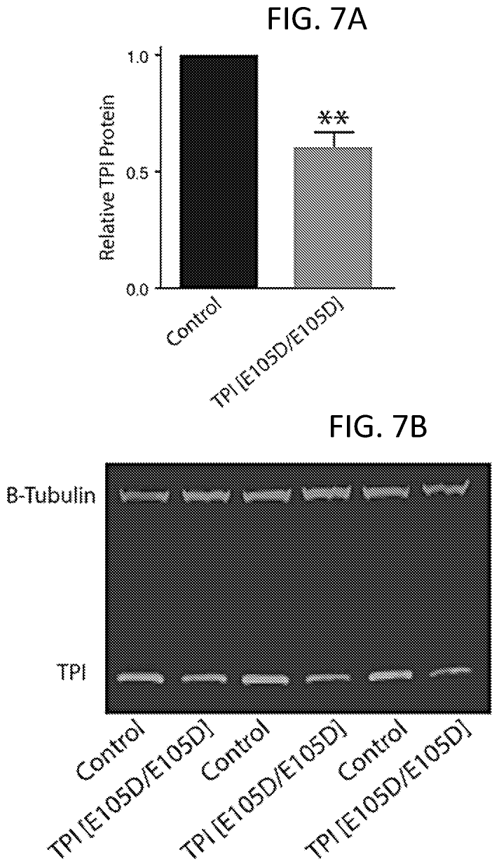

One diagnostic hallmark of TPI Df is a reduction in TPI activity that is almost always first discovered by measuring TPI activity from a blood sample. Pathogenesis and the reduced activity observed in blood samples in TPI Df patients is tied to reductions in TPI protein due to its reduced stability (Roland et al., Biochim Biophys Acta Mol Basis Dis 1865:2257-2266, 2019; Orosz et al., IUBMB Life 58:703-715, 2006). Using patient fibroblasts with the homozygous common mutation, TPI levels were investigated by Western blot as an indicator of protein stability. The results demonstrated a ˜40% reduction in TPI protein relative to the β-tubulin loading control in patient versus control fibroblasts (FIGS. 7A-7B). TPI^{E105D/E105D} (FB104) cells have 0.6252 (SEM=0.05952) relative TPI compared to 1.0 for the normalized control fibroblast levels, which represents a 37.5% decrease in TPI protein. These results are consistent with previously published data on TPI^{E105D} suggesting that the mutant protein is temperature-sensitive and has reduced stability due to proteolysis (Torres-Larios, J Biol Chem 283:23254-23263, 2008; De La Mora-De La Mora et al., Biochim Biophys Acta 1834:2702-2711, 2013).

Elevation of TPI in Patient Cells with Drug Treatment

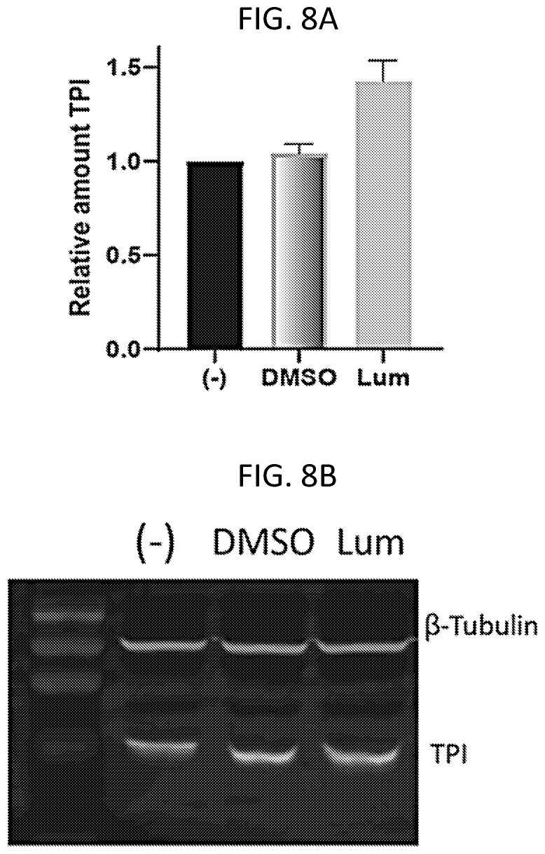

As reduced stability of TPI protein underlies TPI Df pathogenesis and mutant proteins retain activity, altering the turnover of mutant TPI protein may be of therapeutic value. As reduced protein stability has been observed in numerous pathogenic variants of TPI Df including the common mutation, such an approach could be broadly beneficial to TPI Df patients. In this and previous studies in flies, when Hsp90 levels were decreased with RNAi or p-element disruption, the result was an increase in TPI^{sugarkill} levels (Hrizo and Palladino, Neurobiol Dis 40:676-683, 2010). Therefore, if the role of Hsp90 in TPI protein stability translated to human cells, a corresponding increase in TPI^{E105D} levels was expected in response to decreased Hsp90 activity due to pharmacological inhibition. For these studies, TPI^{E105D/E105D} patient fibroblasts (FB104) were examined for changes in TPI protein steady state levels in response to treatment with the Hsp90 inhibitor Luminespib for 48 hours. A corresponding increase in TPI^{E105D} stability was observed in response to treatment with the Hsp90 inhibitor (FIGS. 8A-8B). TPI^{E105D/E105D} (FB104) cells treated with 200 μM Luminespib have relative TPI protein levels of 1.425 (SEM=0.11) compared to 1.043 (SEM=0.05) for DMSO-treated cells, or a 36.6% increase in mutant TPI protein levels. This indicates that the chaperones and quality control factors identified in Drosophila are additional targets for development of TPI deficiency therapeutic treatments.

### Discussion

TPI Df is a devastating recessive disease with early childhood-onset that is caused by numerous missense mutations in the TPI1 gene. Studies using patient cells as well as research in Drosophila, suggest that pathogenesis is driven by mutations that destabilize the protein (Hrizo and Palladino, Neurobiol Dis 40:676-683, 2010; Torres-Larios, J Biol Chem 283:23254-23263, 2008; De La Mora-De La Mora et al., Biochim Biophys Acta 1834:2702-2711, 2013; Seigle et al., Genetics 179:855-862, 2008; Roland et al., Biochim Biophys Acta Mol Basis Dis 1865:2257-2266, 2019; Roland et al., J Cell Sci 126:3151-3158, 2013; Orosz et al., IUBMB Life 58:703-715, 2006). Additionally, mutations in TPI that reduce catalytic activity are generally well-tolerated in mice as long as activity isn't completely abrogated and the resulting protein exhibits normal stability (Segal et al., J Inherit Metab Dis 42:839-849, 2019). Collectively these data led to the hypothesis that stabilizing mutant TPI protein is a promising therapeutic approach for TPI Df (Segal et al., J Inherit Metab Dis 42:839-849, 2019; Hrizo and Palladino, Neurobiol Dis 40:676-683, 20101; Seigle et al., Genetics 179:855-862, 2008). Mutant TPI is degraded by the proteasome in a chaperone-dependent manner (Celotto et al., Genetics 174:1237-1246, 2006; Hrizo and Palladino, Neurobiol Dis 40:676-683, 20101; Seigle et al., Genetics 179:855-862, 2008). Specifically, the role of Hsp70 and Hsp90 in TPI^{sugarkill} degradation has been elucidated, and impairing this degradation led to improvements in progressive neurodegenerative phenotypes (Hrizo and Palladino, Neurobiol Dis 40:676-683, 2010). Existing compounds that target Hsp70 and Hsp90 have been evaluated for treatment of protein folding disorders such as cystic fibrosis and various types of cancer (Brodsky and Chiosis, Curr Top Med Chem 6:1215-1225, 2006; Murphy, Carcinogenesis 34:1181-1188, 2013; Wang and McAlpine, Org Biomol Chem 13:3691-3698, 2015; Ihrig and Obermann, SLAS Discov 22:923-928, 2017; Yuno et al., “Clinical evaluation and biomarker profiling of Hsp90 inhibitors,” In Chaperones: Springer, pages 423-441, 2018). However, Hsp70 and Hsp90 are highly and ubiquitously expressed, promiscuous chaperones that regulate numerous important cellular processes and their inhibitors have the potential for significant side effects (Yuno et al., “Clinical evaluation and biomarker profiling of Hsp90 inhibitors,” In Chaperones: Springer, pages 423-441, 2018; Miyata, Curr Pharm Des 11:1131-1138, 2005). Identifying more specific regulators of TPI degradation may allow for a combinatorial therapy to be developed that will aid in reducing the degradation of TPI, with less toxicity.

The goal of the RNAi screen described herein was to identify novel factors that impact the degradation of mutant TPI, in particular proteins other than HSP70 and HSP90. TPI^{sugarkill} is a relatively unique protein quality control substrate; it is an endogenous mutant protein that is structurally normal, functional and not aggregation prone, yet is still identified by the quality control system of the cell and targeted for proteasomal degradation (Hrizo and Palladino, Neurobiol Dis 40:676-683, 20101; Seigle et al., Genetics 179:855-862, 2008; Roland et al., Biochim Biophys Acta Mol Basis Dis 1865:2257-2266, 2019; Roland et al., J Cell Sci 126:3151-3158, 2013). Therefore, the regulatory network involved in mutant TPI degradation, particularly those proteins that are more specific to mutant TPI, may be ideal targets for pharmacological intervention as potential therapies for TPI deficiency. The screen identified 25 targets, including the ubiquitination pathway, Hsp20 and the RAC complex.

Mitochondrial dysfunction has been implicated in a multitude of myodegenerative and neurodegenerative diseases, and mitochondrial health appears to play an important role in TPI deficiency. Mitochondrial generated oxidative stress has been identified as a contributor to the neurological phenotypes observed in TPI^{sugarkill} flies (Hrizo et al., Neurobiol Dis 54:289-296, 2013). The screen identified three proteins (CG32727, CG4164, and mge) associated with mitochondrial function and it may be of interest to determine the mechanism by which the down regulation of these factors results in TPI stability. Modulation of oxidative stress by diet, such as the ketogenic diet, and by pharmacological methods such as reducing agents, improve phenotypes associated with metabolic mutations in flies (Hrizo et al., Neurobiol Dis 54:289-296, 2013; Fogle et al., J Neurogenet 30:247-258, 2016; Fogle et al., Mol Genet Metab 126:439-447, 2019). This suggests that compounds that modulate mitochondrial function and redox stress may also be of interest in developing a treatment strategy for TPI deficiency patients.

Studies with cell lines derived from TPI^{E105D} patients indicate that the TPI^{E105D} protein has lower steady state levels suggesting it too is unstable (FIGS. 7A-7B) and the reduced TPI protein levels can be “rescued” with an Hsp90 inhibitor (FIGS. 8A-8B). Of course, a 36.6% increase in a protein that is decreased 37.5% from that of wild type does not bring protein levels back to completely normal levels. Such an increase would return TPI protein levels to ˜85% of their normal or wild type levels ((1.366×0.625)×100%=85.38%). Although this is not considered a complete “rescue”, such an improvement would be predicted to be of immense therapeutic value. Therefore, these data demonstrate using patient cells that inhibiting the degradation pathway of mutant TPI protein can be beneficial as a therapeutic approach for TPI Df treatment. Luminsepib and other HSP90 inhibitors would not likely make viable TPI Df treatments as they are generally not well tolerated and the ones tested so far do not have a toxicity profile needed for chronic treatment in humans. However, several of the other proteins identified in this screen as putative modulators of mutant TPI turnover are likely much less promiscuous (e.g., more selective) than HSP90 and represent viable targets for therapeutic intervention using inhibitors. Furthermore, inhibition of a more TPI-selective protein quality control target will likely be much better tolerated as a TPI Df therapy, which requires chronic and likely life-long administration.

### Example 3: Materials and Methods

This example describes the materials and experimental procedures for the studies described in Example 4.

### Generation and Culturing HEK293 TPI^{E105D-GFP} Cells

HEK293 cells expressing a mutant (E105D) TPI-GFP fusion protein were created using the Flp-In System (Invitrogen, Catalog #K6010-01 and K6010-02) following the manufacturer's protocol. Briefly, a host cell line expressing the transfected Flp-In target site vector, pFRT/lacZeo, was obtained following zeocin resistance selection. TPI^{E105D-GFP} fusion construct was cloned into the pcDNA 5/FRT expression vector. Co-transfection of 0.1 μg of the pcDNA 5/FRT construct (390 ng/μL) and 0.9 μg of the Flp recombinase expression vector (pOG44, 127 ng/μL) into the Flp-In host cell line was performed with 3.75 μL Lipofectamine 2000 reagent (Invitrogen). A hygromycin dose response was performed to determine the minimum concentration of hygromycin B required to kill untransfected host cells. Clones of cells were selected that exhibited hygromycin B resistance. Expression of the TPI^{E105D-GFP} fusion protein was verified via Western blot. The cells were cultured using standard methods (37° C., 5% CO₂) in normal media (DMEM with 10% serum, 100 U penicillin/100 μg streptomycin/ml (Lonza)). To document subcellular localization of mutant GFP-tagged TPI, nuclei were stained with Hoechst 33342 and imaged on an ImageXpress Ultra confocal high content reader in the UV and GFP channel, respectively. A z-series of images (60× objective, 90 planes, 0.3 μm z-steps) was acquired in both channels, and the color combined stack subjected to 3D reconstruction in MetaXpress. A single slice of the reconstructed image was chosen to illustrate the phenotype of mutant TPI-GFP expression.

### Generation and Culturing U2OS TPI^{E105D-GFP} Cells

U2OS osteosarcoma cells (ATCC HTB-96) engineered to express the mutant (E105D) TN-GFP protein were generated using the identical method described above for HEK cells and maintained in McCoy's 5A medium (ThermoFisher 16600082).

### Compounds

A full list of compounds used in the studies disclosed herein is provided in Table 4.

TABLE 4
Drug information

| | Stock | | | | | | | | | | |
|---|---|---|---|---|---|---|---|---|---|---|---|
| Compound | Concentration | | Solvent | Mol. Weight | | Class | Amount | | Form | Company | Cat. # |
| geldanamycin | 50 | mM | DMSO | 560.64 | g/mol | Hsp90 inhib | 225 | mg | solid | Fisher | 50-148-646 |
| MG132 | 10 | mg/mL | DMSO | 475.62 | g/mol | Proteasome | 5 | mg | crystalline | Fisher | NC9819784 |
| | | | | | | inhib | | | solid | | |
| withaferin a | 1 | mg/ml | methanol | 470.60 | g/mol | Hsp90 inhib | 5 | mg | powder | sigma | W4394- |
| | | | | | | | | | | | 5MG |
| celastrol | 50 mM | | DMSO | 450.61 | g/mol | Hsp90 inhib | 10 | mg | red solid | Sigma | C0869- |
| | (5 mg/.2219 mL) | | | | | | | | | | 10MG |
| bortezomib | 50 mM | | DMSO | 384.24 | g/mol | proteaseome | 5 | mg | solid | Sigma | 5043140001 |
| | (5 mg/.2603 mL) | | | | | inhib | | | | | |
| cycloheximide | 10 | mg/ml | water | 281.35 | g/mol | translation | 5 | g | irregular shaped | Sigma | C4859-1ML |
| | | | | | | inhib | | | granules | | |
| tanespimycin | 50 mM | | DMSO | 585.69 | g/mol | Hsp90 inhib | 500 | μg | solid | Sigma | A8476- |
| | (5 mg/.1707 mL) | | | | | | | | | | 500UG |
| luminespib | 50 mM | | DMSO | 465.55 | g/mol | Hsp90 inhib | 5 | mg | powder | Selleckchem | S1069 |
| | (5 mg/.2148 mL) | | | | | | | | | | |
| Ver-155008 | 50 mM | | DMSO | 556.40 | g/mol | Hsp70 inhib | 5 | mg | powder | Sigma | SML0271- |
| | (5 mg/.1797 mL) | | | | | | | | | | 5MG |
| Resveratrol | 200 | mM | DMSO | 228.247 | g/mol | natural | 1 | g | yellow crystal- | Fisher | R00711G |
| | | | | | | | | | powder | | |
| Itavastatin | 10 | mM | DMSO | 440.49 | g/mol | statin | 5 | mg | white to beige | Sigma | SML2473- |
| | | | | | | | | | powder | | 5MG |

### NIH Clinical Collection

The NIH Clinical Collection is a plated array of 446 small molecules that encompasses FDA approved drugs and agents that have a history of use in human clinical trials. The collection was assembled by the National Institutes of Health (NIH) through the Molecular Libraries Roadmap Initiative as part of its mission to enable the use of compound screens in biomedical research and is maintained as assay ready daughter plates at the University of Pittsburgh Drug Discovery Institute (UPDDI).

### LOPAC Library

The Library of Pharmacologically Active Compounds (LOPAC) was obtained from Sigma-Aldrich. It contains 1280 biologically annotated small molecules and is the standard assay validation test set at the UPDDI.

Assay Development and Prescreen with Selected PQC Inhibitors

HEK293 TPI^{E105D-GFP} cells were plated (50,000 cells/well in collagen-coated 96-well thin-bottom microplates and allowed to attach overhight. Cells were treated with four point, two-fold gradients of 5× treatment solutions of test agents (6 replicates per condition). The final concentration of vehicle was 0.2%. After 48 hours in culture, cells were preincubated with propidium iodide (PI) for 30 minutes and imaged live in the GFP and Texas Red channels on an ImageXpress Ultra high content reader (Molecular Devices) using a 20× objective Images were analyzed with the multiwavelength cell scoring application using GFP to define and enumerate cells. The integrated GFP intensity per cell was used to quantify mutant TPI expression; the number of cells was used as a proxy for growth inhibition and/or cell loss; and the percentage of cells that exceeded a threshold for PI based on vehicle-treated controls was used to measure acute toxicity (necrosis).

### Flow Cytometry

HEK293T cells with GFP-TPI (E105D) mutation were plated (50,000/well) in the wells of a 96-well plate (100 μl) and treated with 25 μl of a 5× treatment solution of luminespib for 48 hours. Cells were detached by adding 25 μl 17.5 mM EDTA, pH 6.15 for 2 hours (Kaur and Esau, Biotechniques 59(3):119-126, 2015) directly to growth medium (final concentration 2.5 mM). Plates were subsequently stained with 25 μl PI (5 μg/ml in PBS), dislodged by pipetting, and analyzed by flow cytometry. Samples were acquired on a BD Fortessa SORP cytometer (BD Biosciences). Dislodged (resuspended) cells were run on the BD High Throughput Sampler (HTS) with the following settings to ensure even sampling: Sample flow rate: 1.5 μl/second, Sample volume: 80 μl, Mix volume: 40 μl (40% total volume), Mix speed: 200 μl/second, Number of mixes: 4, Wash volume: 400 μl. An attempt was made to collect at least 25,000 events per sample. Forward scatter (FSC), Side scatter (SSC), GFP (488_515_30) and PI (561_582_15) detectors were optimized on untreated viable HEK293T cells. Samples were analyzed for expression of GFP and viability (PI negative events). Several population patterns were observed and correlation with FSC/SSC revealed the following states: viable (live), early apoptotic and late apoptotic cells.

### Pilot Screen Using the NIH Clinical Collection and LOPAC Library

Assay ready daughter plates of the NIH Clinical Collection containing 2 μL of a 1 mM DMSO stock were reconstituted in DMEM (60 μL) to yield a 30 μM intermediate treatment concentration. 60 μl of DMSO (3%) was added to columns 1 and 24, and 60 μL of 600 nM luminespib was added to columns 2 and 23 to serve as negative and positive controls, respectively. Aliquots (15 μl) were transferred directly to cells in 384 well plates using an Agilent Bravo liquid handling robot (final concentration in assay, 10 μM). For the 1 μM condition, an aliquot of the 30 μM treatment solution was diluted 1:10 and controls added as above. Aliquots (15 μl) were transferred to cells to yield a 10 μM final concentration. Plates were centrifuged for 30 seconds at 200×g and incubated at 37° C. After 24 hours, plates were imaged live in the GFP channel and returned to the incubator; after 48 hours cells were stained with PI, imaged live in the GFP and PI channels, and analyzed as described above. The LOPAC library was screened using the same procedure except that only the 10 μM, 48 hour condition was used.

### Culturing Patient Fibroblasts

Patient fibroblasts were obtained from a male TPI deficiency patient via skin punch. The cells were de-identified and are known only as FB104. The cells are homozygous for the common E105D mutation (also known as E104D in legacy publications). Fibroblasts were cultured using standard methods (37° C., 5% CO₂) in complete media (DMEM with 10% serum, 100 U penicillin/100 μg streptomycin/ml (Lonza), 2 mM L-glutamine (Gibco) and supplemental non-essential amino acids (Gibco, Carlsbad, CA)).

### Western Blotting

FB104 patient fibroblasts [E105D/E105D] treated with luminespib (200 nM), resveratrol (100 μM), itavastatin (5 μM), or DMSO (0.1%, Sigma Aldrich) were trypsinized (0.05% for 5 minutes), pelleted, resuspended in RIPA buffer with protease inhibitors (100 μM PMSF, 1 μg/μL leupeptin, 0.5 μg/μL pepstatin A) and pulse sonicated. Protein concentrations were determined using a BCA assay (Pierce) Immunoblotting was performed on whole protein cell lysates following the addition of an equal volume of 2×SDS PAGE sample buffer (4% SDS, 4% β-mercaptoethanol, 130 mM Tris HCl pH 6.8, 20% glycerol). Proteins were resolved by SDS-PAGE (12%) and transferred onto 0.45 μm PVDF membrane. The blots were blocked in Odyssey Blocking Buffer (Licor) and incubated with anti-TPI (1:5000; rabbit polyclonal FL-249; Santa Cruz Biotechnology sc-30145) or anti-beta-tubulin (1:1000; mouse polyclonal E7-C; Developmental Studies Hybridoma Bank, Iowa City, IA) diluted in Odyssey Blocking Buffer (Licor). Following washes in PBST, the blots were incubated with anti-mouse-IR800 (Fisher Scientific) and anti-rabbit-IR680 (Molecular Probes) both diluted to 1:20,000 in 0.1% Tween® 20 blocking buffer. Blots were washed in PBST and developed using Odyssey Infrared Imaging System. Quantification of the scanned images was performed digitally using the Image Studio Ver 5.2 software. TPI levels were normalized to beta tubulin loading control and differences in TPI expression were evaluated by a two-tailed Student's t-test.

### High-Content Analysis of TPI Levels in Patient Fibroblasts by Immunocytochemistry

FB303 cells were plated (1000/well) in a 384 well collagen-coated microplate. Cells were treated with vehicle (DMSO) or ten-point, two-fold gradients of test compounds for 48 hours in quadruplicates. Cells were fixed with 4% formaldehyde and immunostained with an anti-TPI antibody (Santa Cruz sc-30145) followed by a Cy3-conjugated goat anti-rabbit secondary (Jackson Immunoresearch). Nuclei were counterstained with Hoechst 33342 Images were acquired on an OPERA Phenix high-content imager (20× air objective, widefield, 4 fields/well) and analyzed for total cellular GFP intensity (HEK and U2OS) or total cellular Cy3 intensity (FB303) using Harmony 5.0 image analysis software (Perkin Elmer).

### Three-Day Variability

Two full 384-well microplates of HEK293 TPI^{E105D-GFP} cells (10,000 cells/well) were treated with vehicle (1% DMSO) or luminespib (200 nM) on three consecutive days using a Perkin Elmer MDT or Agilent Bravo liquid handler. After a 4-hour incubation, cells were imaged live in the GFP channel as described above and analyzed for TPI-GFP expression using the MetaXpress multiwavelength scoring application. Assay performance was assessed between plates and between days by calculating signal-to-background ratios (S/B), coefficients of variation (CV), and Z-factors, as described (Iversen et al., HTS Assay Validation, in Assay Guidance Manual, G. S. Sittampalam, et al., Editors. 2004, Eli Lilly & Company and the National Center for Advancing Translational Sciences: Bethesda (MD)).

### Example 4: A High Content Screening Assay for Small Molecules that Stabilize Mutant Triose Phosphate Isomerase (TPI) as Treatments for TPI-Deficiency

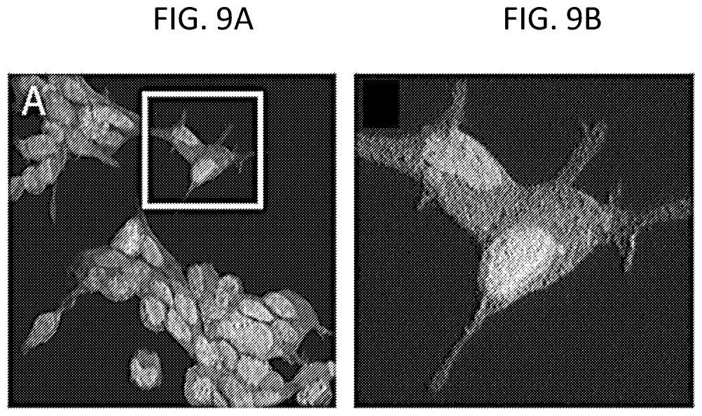

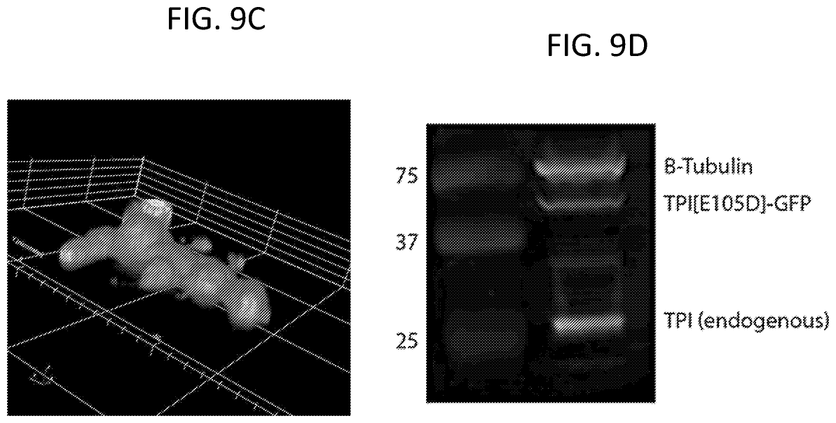

The “common” TPI^{E105D} mutation is well-characterized biochemically and structurally, and results in an ˜50% reduction in steady state protein levels in patient cells. Described in this Example is the development of an HEK TPI^{E105D-GFP} stable knock-in cell line using the Thermo-Fisher Flp-In system. In compound heterozygotes, the degradation of each mutant protein occurs independently (Roland et al., PLoS Genet 12(3): e1005941, 2016); thus, mutant GFP-tagged TPI degradation was not significantly affected by wild type TPI that keeps the cells healthy. HEK TPI^{E105D-GFP} cells provide a convenient optical assay of steady state mutant human TPI protein in the context of a human cell. FIGS. 9A-9C show stable and uniform expression of mutant GFP-tagged TPI that is consistent with a ubiquitous nuclear and cytosolic localization, suggesting it could serve as a discovery assay for compounds that increase levels of mutant TPI.

### Assay Development

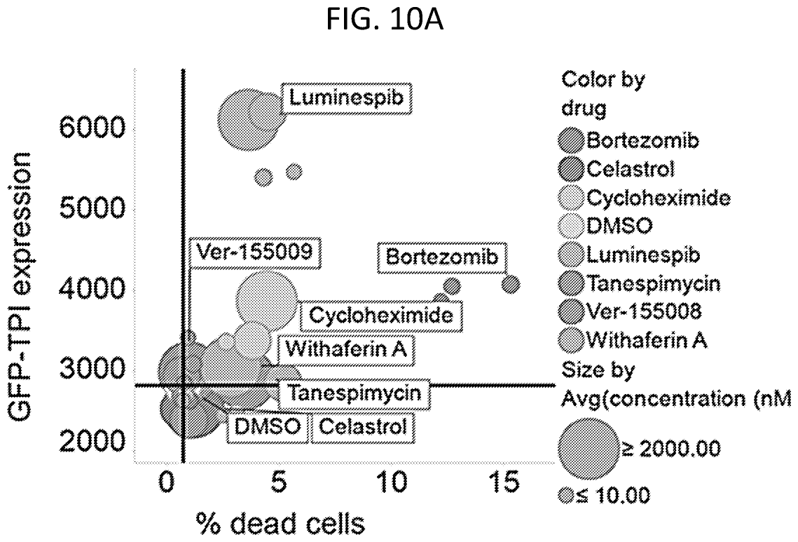

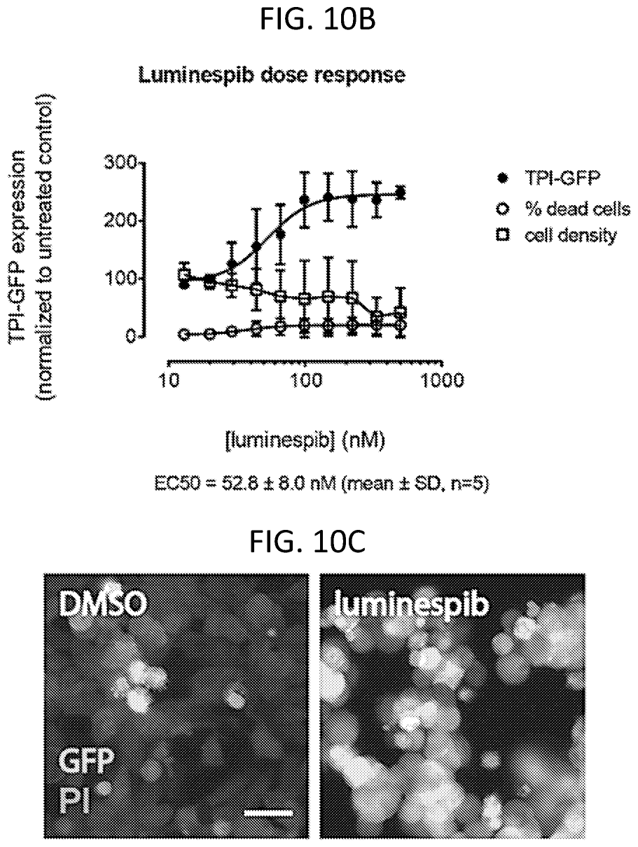

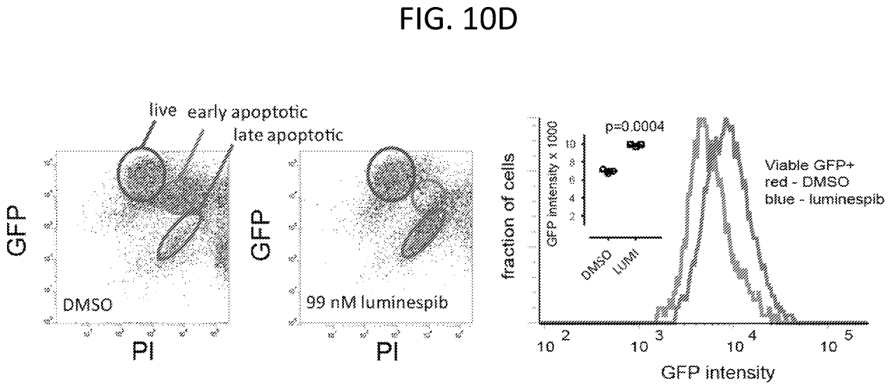

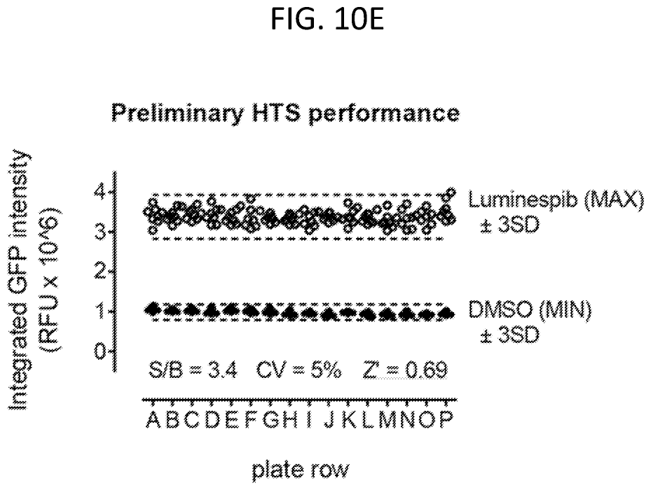

A panel of inhibitors known to target mutant TPI for degradation in Drosophila were tested and their effect on HEK TPI^{E105D-GFP} cells was examined via confocal microscopy. Consistent with Drosophila results, bortezomib, cycloheximide and luminespib all showed a significant increase in TPI^{E105D-GFP} fluorescence, demonstrating that TPI^{E105D} turnover in human cells is similarly regulated as TPI^{sgk} protein in Drosophila, as well as confirming the utility of the reporter system (FIG. 10A). All agents in this class significantly inhibited cell growth and displayed some acute toxicity as measured by propidium iodide (PI) staining (FIG. 10A). Of the seven agents tested, luminespib produced the highest fold increase in GFP with the lowest toxicity (FIGS. 10A, 10B). Due to the toxicity, it was desirable to ensure the increase in GFP was the result of more TPI^{E105D-GFP} in the live cells and not an artifact of the dying cells. Fluorescence micrographs indicated that PI positive cells expressed no GFP (FIG. 10C). To confirm these results and to eliminate the possibility that morphological changes could contribute to the enhanced GFP levels, flow cytometry, which measures total protein content independent of cell morphology, was performed. Luminespib caused apoptosis, but the GFP increase observed was exclusively from the healthy cells, further validating the use of this human TPI^{E105D-GFP} reporter (FIG. 10D). A pilot test was conducted in 384-well plates using a meaningful number of replicates (n=144) with luminespib as a positive control. The results demonstrated that the method met universally accepted HTS standards, demonstrating the viability of this screening method and the utility of luminespib as a positive control (FIG. 10E).

### Compound Pilot Screen

A pilot library screen was performed using the NIH Clinical Collection in the HEK TPI^{E105D-GFP} cell line. The NIH Clinical Collection maintained at the UPDDI contains 446 FDA approved drugs and small molecules that have a history of use in human clinical trials. The collections were assembled through the Molecular Libraries and Imaging Initiative as part of its mission to enable the use of compound screens in biomedical research. To extract as much information from this library as possible, the library was screened in duplicate at two concentrations (10 μM and 1 μM) and at two time points (24 hours and 48 hours). Cells were plated in collagen-coated 384-well plates and treated the next day with library compounds in duplicate using the Agilent Bravo pipetting robot. DMSO (1%) and luminespib (200 nM) served as negative (MIN) and positive (MAX) controls (n=32 each). Cells were incubated at 37° C., scanned on the ImageXpress Ultra high content reader after 24 hours, and returned to the incubator for an additional 24 hours. Thirty minutes before the 48-hour scan, cells were stained with PI and imaged in the GFP and Texas Red channels to assess TPI^{E105D-GFP} levels, loss of cell attachment, and acute toxicity (necrosis). Plates were analyzed with the multi-wavelength cell scoring application in MetaXpress; TPI levels were calculated on a per cell basis. Cell densities and TPI^{E105D-GFP} expression were then normalized to DMSO control.

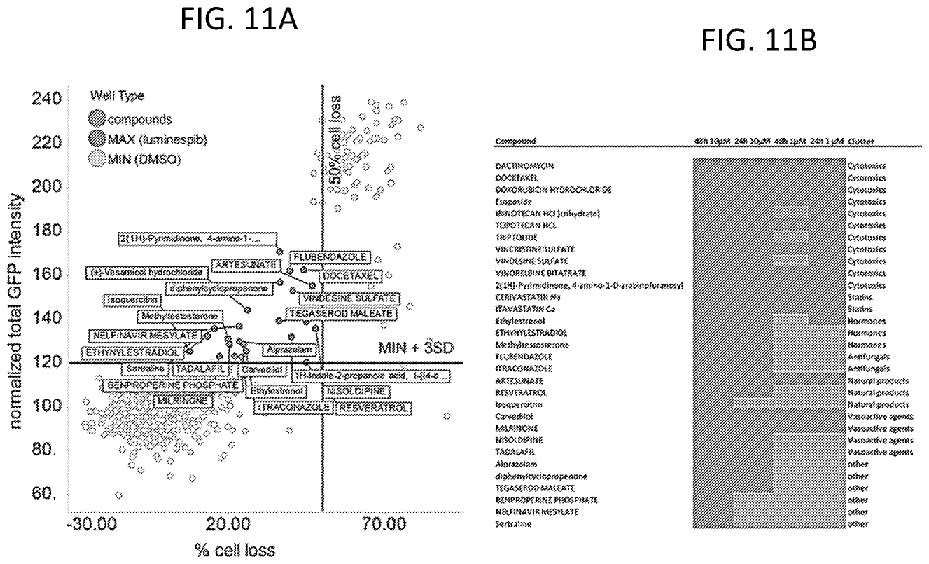

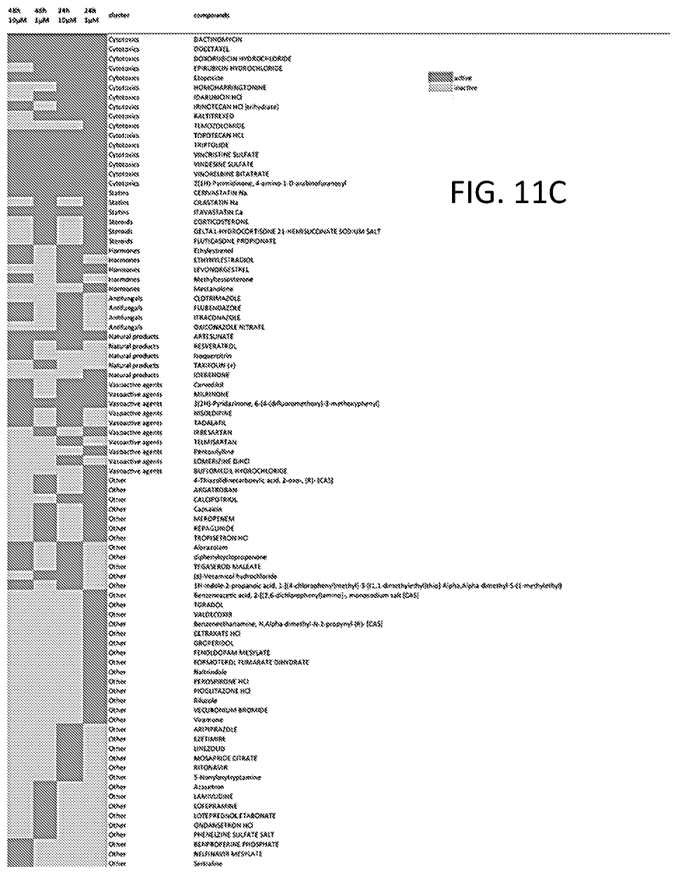

The performance of the HEK TPI^{E105D-GFP} cellular assay under conditions of compound screening was excellent (FIGS. 11A-11C). Total cellular mutant TPI (GFP) was used as the primary screen parameter with luminespib (200 nM) as a positive control. Intra-plate S/B values and Z-factors were 1.64±0.11 and 0.30±0.22 on day 1, and 2.16±0.98 and 0.51±0.06 on day 2, respectively. This suggested a 48-hour incubation was necessary for optimal assay performance. There was good agreement between replicates (r²=0.88). The 10 μM/48 h condition identified 31 compounds that elevated TPI^{E105D-GFP} by more than 3 standard deviations (SD) over the mean of the negative controls (a pseudo z-score of >3, FIG. 11A, y-axis). Among these were eleven antineoplastic agents (FIG. 11B), which is not surprising as the screen is using transformed cells and the NIH Clinical Collection contains a disproportionately large number of anticancer agents. The majority of these agents could be eliminated by measurements of cell loss (FIG. 11A, x-axis) and/or PI staining and were excluded from further analysis. A comprehensive heatmap of all positive compounds that were active at any condition is shown in FIG. 11C.

The remaining 20 hits contained several clusters, namely statins (2 compounds), hormones (3), antifungals (2), natural products (3), vasoactives (4), and 5 singletons with diverse mechanism of action. Of these, 16 (80%) repeated at the 10 μM condition at the earlier time point, and five repeated in every condition (itavastatin, cerivastatin, artesunate, carvedilol, milrinone). Compounds that did not repeat were mostly singletons. Although vasoactive agents in general showed high repeat rates, from a translational perspective, they would likely not be useful for the treatment of an early childhood metabolic disease. After these considerations, two classes of agents looked most interesting—statins and natural products. In particular, resveratrol and itavastatin had properties that could make them candidates for repurposing. Resveratrol as a nutritional supplement would represent a near immediately applicable treatment for current TPI Df patients. A review of the clinical literature revealed that resveratrol had beneficial effects on skeletal muscle (Always et al., J Gerontol A Biol Sci Med Sci 72(12):1595-1606, 2017), possibly by attenuating oxidative stress in myoblasts (Haramizu et al., J Nutr Biochem 50:103-115, 2017). This could be highly relevant as muscle wasting is a hallmark of TPI-Df. Statins have been associated with modulation of heat shock proteins (Forouzanfar et al., Pharmacol Res 134:134-144, 2018), although the clinical picture is heterogeneous. However, in the right context it is conceivable that statins could mediate protein stability through HSP modulation. While the doses of statins that are effective in cardiovascular disease are usually far lower than those observed in the laboratory for other biological activities, itavastatin has been shown to reach peak plasma levels in humans that correlate with concentrations needed to increase TPI^{E105D-GFP} in HEK cells (0.55 μM) (Luo et al., Curr Ther Res Clin Exp 77:52-57, 2015).

### Library of Pharmacologically Active Compounds (LOPAC) Screen

Resveratrol and mevastatin were positive hits in an independent screen of the LOPAC using the HEK293 TPI^{E105D-GFP} cell line. The LOPAC library (Sigma-Aldrich) contains 1280 biologically annotated small molecules. Screening conditions were identical to those described for the NIH Clinical Collection, except that the screen was performed only at the 10 μM, 48 hour condition.

### Hit Validation

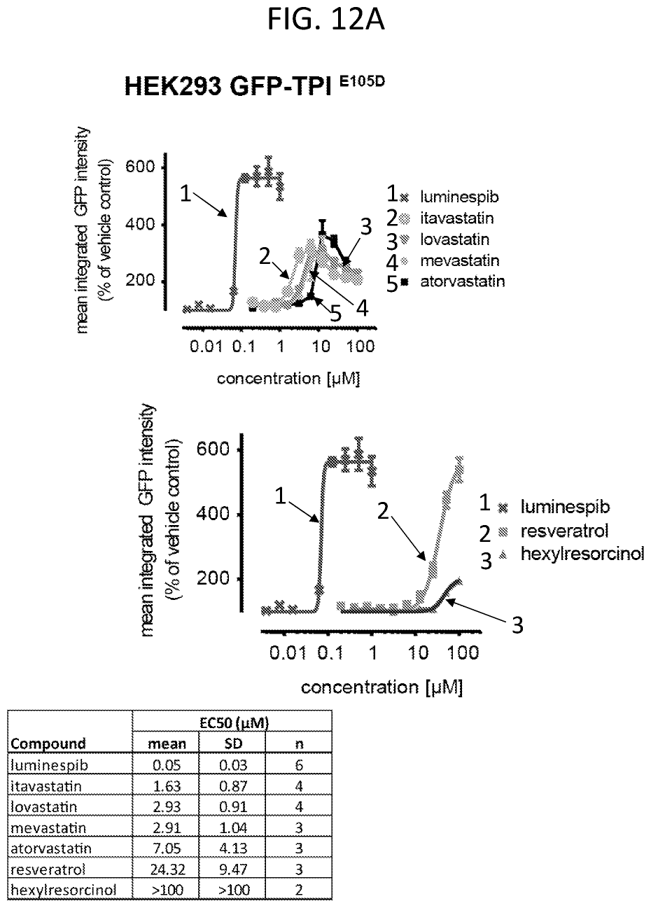

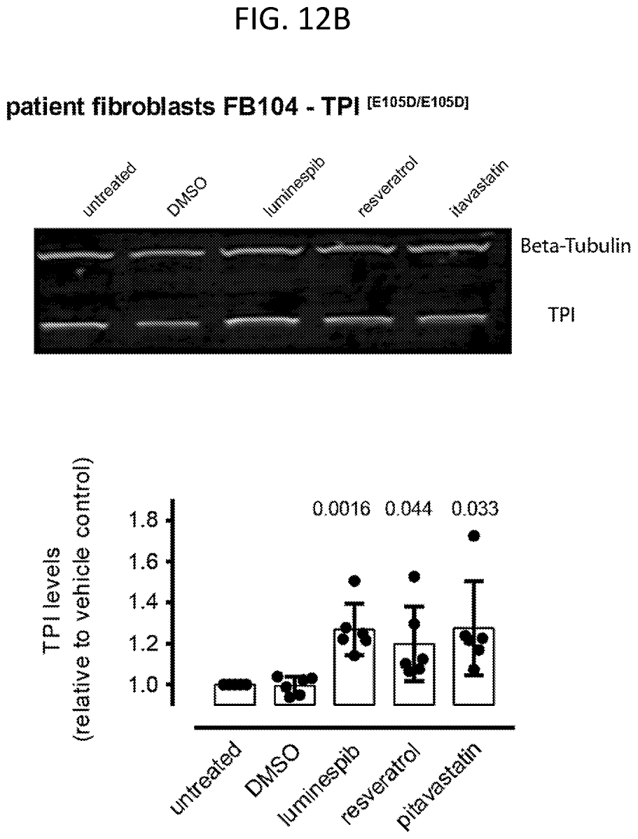

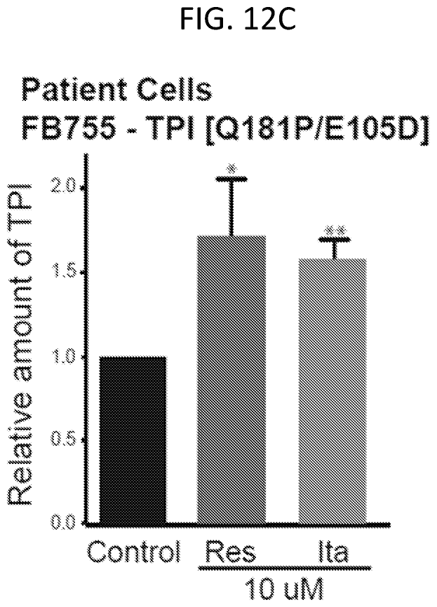

Using fresh samples repurchased from commercial suppliers, both resveratrol and itavastatin showed concentration-dependent increases in TPI^{E105D-GFP} (FIG. 12A). Western blots using fibroblasts from TPI^{E105D} and TPI^{Q181P} Df patients confirmed elevated mutant TPI levels by luminespib using an orthogonal non-imaging method (FIGS. 12B, 12C). These data document that the HEK TPI^{E105D-GFP} assay meets HTS performance criteria and can identify low toxicity, bona fide TPI inducers/stabilizers with different mechanisms of action, which are ready for validation in Drosophila and in a mouse TPI Df animal model. The data also validate a secondary hit confirmation paradigm consisting of complementary orthogonal assays in patient cells.

### Confirmation of Hits

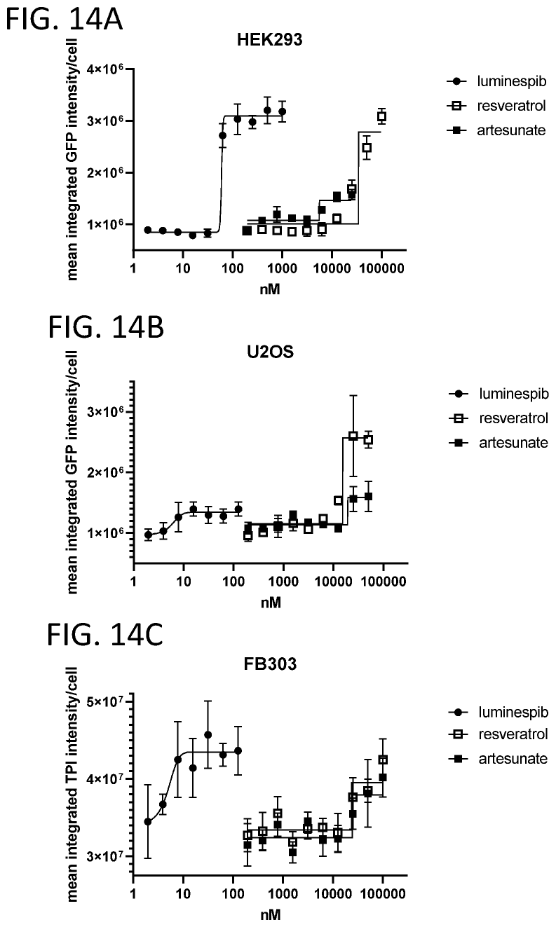

Dose-response confirmation of hits from the NIH clinical collection by high-content analysis confirmed activity of resveratrol and artesunate in an additional TPI-GFP cell line (U20S-TPI-E105D) and patient fibroblasts. The results are shown in FIGS. 14A-14C.

### Multi-Day Variability Assessments

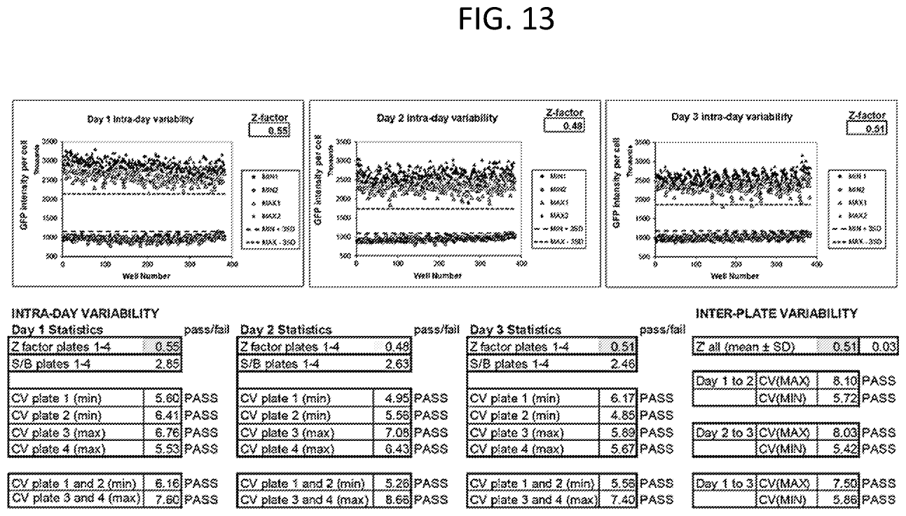

Based on the assay development and validation results above, a formal three-day variability study was performed, as is standard for HTS implementation of HCS assays at the UPDDI (Vogt and Lazo, Methods 42(3):268-277, 2007; Joy et al., PLoS One 9(2):e88350, 2014; Poe et al., J Biomol Screen 19(4):556-565, 2104; Nmezi et al., SLAS Discov 25(8):939-949, 2020). Two full 384-well microplates of HEK TPI^{E105D-GFP} cells were plated and treated on three consecutive days with 1% DMSO (MIN) or 200 nM luminespib (MAX) using a multidrop bulk liquid despenser and a Perkin Elmer Janus MDT pipetting robot. After a 48-hour incubation, cells were imaged on an ImageXpress Ultra high-content reader and analyzed for TPI-GFP expression. Mean, Standard deviation (SD), coefficients of variance (CV), signal-to-background ratios (S/B), and Z-factors (Zhang et al., J Biomol Screen 4(2):67-73, 1999) were calculated for each plate, each day of experiments, and across days. Scatter plots illustrate day-to-day performance; tables show calculated Intra-plate and inter-plate variability statistics (FIG. 13). The assay met accepted HTS criteria for intra-plate and inter-day variability on all three days (CV<10%). The mean Z-factor across all three days was 0.51±0.03, conforming to accepted HTS standards, and documenting HTS readiness.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims. We therefore claim all that comes within the scope and spirit of these claims.

1. A method for identifying an agent that promotes stability of a mutant form of the human triosephosphate isomerase (TPI) protein, comprising:
culturing in a cell culture vessel cells stably expressing a fusion protein comprising the mutant TPI protein and a fluorescent protein;
contacting the cells with a candidate agent; and
measuring fluorescence intensity of the cells, wherein an increase in fluorescence intensity of the cells compared to control cells cultured in the absence of the candidate agent, identifies the candidate agent as an agent that promotes stability of the mutant TPI protein.

2. The method of claim 1, wherein the mutant TPI protein comprises:
a glutamic acid to aspartic acid substitution at position 105 (TPI^{E105D});
a valine to methionine substitution at position 232 (TPI^{V232M});
a glutamine to proline substitution at position 181 (TPI^{Q181P});
an arginine to glutamine mutation at position 190 (TPI^{R190Q});
a cysteine to tyrosine substitution at position 42 (TPI^{C42Y});
an alanine to aspartic acid substitution at position 63 (TPI^{A63D});
a glycine to alanine substitution at position 73 (TPI^{G73A});
a glycine to arginine substitution at position 123 (TPI^{G123R});
a valine to methionine substitution at position 155 (TPI^{V155M});
an isoleucine to valine substitution at position 171 (TPI^{I171V});
a phenylalanine to leucine substitution at position 241 (TPI^{F241E}); or
a phenylalanine to serine substitution at position 241 (TPI^{E241S}),
wherein amino acid numbering is based on SEQ ID NO: 1.

3-5. (canceled)

6. The method of claim 1, wherein the cell culture vessel is a multi-well plate and/or the cell culture vessel is coated in collagen.

7-8. (canceled)

9. The method of claim 1, wherein the candidate agent is a small molecule.

10. The method of claim 1, wherein measuring fluorescence intensity comprises optical detection.

11. The method of claim 1, wherein the method is a high-throughput method, wherein the cell culture vessel is a multi-well plate and fluorescence intensity is measured optically using a multi-well plate reader.

12-13. (canceled)

14. A method of treating a subject who has triosephosphate isomerase deficiency (TPI DO, comprising administering to the subject a therapeutically effective amount of an agent that promotes stability of a mutant form of the human TPI protein.

15. The method of claim 14, wherein the mutant TPI protein comprises:
a glutamic acid to aspartic acid substitution at position 105 (TPI^{E105D});
a valine to methionine substitution at position 232 (TPI^{V232M});
a glutamine to proline substitution at position 181 (TPI^{Q181P});
an arginine to glutamine mutation at position 190 (TPI^{R190Q});
a cysteine to tyrosine substitution at position 42 (TPI^{C42Y});
an alanine to aspartic acid substitution at position 63 (TPI^{A63D});
a glycine to alanine substitution at position 73 (TPI^{G73A});
a glycine to arginine substitution at position 123 (TPI^{G123R});
a valine to methionine substitution at position 155 (TPI^{V155M});
an isoleucine to valine substitution at position 171 (TPI^{I171V});
a phenylalanine to leucine substitution at position 241 (TPI^{F241E}); or
a phenylalanine to serine substitution at position 241 (TPI^{E241S}).

16. (canceled)

17. The method of claim 14, wherein the agent is a small molecule.

18. The method of claim 14, wherein the agent is luminespib, cerivastain, itavastatin, mevastatin, atorvastatin, ethylestrenol, ethynylestradiol, methyltestosterone, flubendazole, itraconazole, artesunate, resveratrol, isoquercitrin, carvedilol, milrinone, nisoldipine, tadalafil, alprazolam, diphenylcyclopropenone, tegaserod maleate, benproperine phosphate, nelfinavir mesylate, or sertraline.

19-20. (canceled)

21. A method of treating triosephosphate isomerase deficiency (TPI Df) in a subject, comprising administering to the subject a therapeutically effective amount of an agent that inhibits expression or activity of aconitase 1 (ACO1), actin related protein 3 (ACTR3), anaphase promoting complex subunit 10 (ANAPC10), anti-silencing function 1A histone chaperone (ASF1A), cell division cycle 34, ubiquitin conjugating enzyme (CDC34), carboxypeptidase B1 (CPB1), crystallin alpha A (CRYAA), cullin 3 (CUL3), dynein axonemal assembly factor 4 (DNAAF4), DnaJ heat shock protein family (Hsp40) member B11 (DNAJB11), DnaJ heat shock protein family (Hsp40) member C2 (DNAJC2), DnaJ heat shock protein family (Hsp40) member C15 (DNAJC15), endoplasmic reticulum protein 29 (ERP29), GTP binding protein 6 (GTPBP6), heat shock protein family B (small) member 1 (HSPB1), HECT, UBA and WWE domain containing E3 ubiquitin protein ligase 1 (HUWE1), MPV17 mitochondrial inner membrane protein like 2 (MPV17L2), proteasome 20S subunit alpha 7 (PSMA7), SEC61 translocon subunit gamma (SEC61G), translocase of outer mitochondrial membrane 22 (TOMM22), ubiquitin conjugating enzyme E2 B (UBE2B), ubiquitin conjugating enzyme E2 Q1 (UBE2Q1), ubiquitin specific peptidase 14 (USP14), ubiquitin specific peptidase 16 (USP16), or ubiquitin specific peptidase 45 (USP45), thereby treating TPI Df in the subject.

22. The method of claim 21, wherein the agent inhibits expression of the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 gene.

23. The method of claim 22, wherein the agent is an antisense compound targeting an ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61 G, TOMM22, UBE2B, UBE2Q1, USP14, USP16 or USP45 nucleic acid.

24. (canceled)

25. The method of claim 21, wherein the agent inhibits activity of a protein encoded by the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61 G, TOMM22, UBE2B, UBE2Q1, USP14, USP16 or USP45 gene.

26. The method of claim 25, wherein the agent is a small molecule inhibitor or a monoclonal antibody.

27-28. (canceled)

29. The method of claim 21, wherein the subject expresses a mutant form of the triosephosphate isomerase (TPI) protein, wherein the mutant TPI protein comprises:
a glutamic acid to aspartic acid substitution at position 105 (TPI^{E105D});
a valine to methionine substitution at position 232 (TPI^{V232M});
a glutamine to proline substitution at position 181 (TPI^{Q181P});
an arginine to glutamine mutation at position 190 (TPI^{R190Q});
a cysteine to tyrosine substitution at position 42 (TPI^{C42Y});
an alanine to aspartic acid substitution at position 63 (TPI^{A63D});
a glycine to alanine substitution at position 73 (TPI^{G73A});
a glycine to arginine substitution at position 123 (TPI^{G123R});
a valine to methionine substitution at position 155 (TPI^{V155M});
an isoleucine to valine substitution at position 171 (TPI^{I171V});
a phenylalanine to leucine substitution at position 241 (TPI^{F241E}); or
a phenylalanine to serine substitution at position 241 (TPI^{E241S}),
wherein amino acid numbering is based on SEQ ID NO: 1.

30. (canceled)

31. A method of promoting stability of a mutant form of the human triosephosphate isomerase (TPI) protein in human cells, comprising contacting the cells with an effective amount of an agent that inhibits expression or activity of aconitase 1 (ACO1), actin related protein 3 (ACTR3), anaphase promoting complex subunit 10 (ANAPC10), anti-silencing function 1A histone chaperone (ASF1A), cell division cycle 34, ubiquitin conjugating enzyme (CDC34), carboxypeptidase B1 (CPB1), crystallin alpha A (CRYAA), cullin 3 (CUL3), dynein axonemal assembly factor 4 (DNAAF4), DnaJ heat shock protein family (Hsp40) member B11 (DNAJB11), DnaJ heat shock protein family (Hsp40) member C2 (DNAJC2), DnaJ heat shock protein family (Hsp40) member C15 (DNAJC15), endoplasmic reticulum protein 29 (ERP29), GTP binding protein 6 (GTPBP6), heat shock protein family B (small) member 1 (HSPB1), HECT, UBA and WWE domain containing E3 ubiquitin protein ligase 1 (HUWE1), MPV17L2, proteasome 20S subunit alpha 7 (PSMA7), SEC61 translocon subunit gamma (SEC61G), translocase of outer mitochondrial membrane 22 (TOMM22), ubiquitin conjugating enzyme E2 B (UBE2B), ubiquitin conjugating enzyme E2 Q1 (UBE2Q1), ubiquitin specific peptidase 14 (USP14), ubiquitin specific peptidase 16 (USP16), or ubiquitin specific peptidase 45 (USP45), thereby promoting stability of the mutant TPI protein.

32. The method of claim 31, wherein the agent inhibits expression of the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 gene.

33. The method of claim 32, wherein the agent is an antisense compound targeting an ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61G, TOMM22, UBE2B, UBE2Q1, USP14, USP16 or USP45 nucleic acid.

34. (canceled)

35. The method of claim 31, wherein the agent inhibits activity of a protein encoded by the ACO1, ACTR3, ANAPC10, ASF1A, CDC34, CPB1, CRAA, CUL3, DNAAF4, DNAJB11, DNAJC2, DNAJC15, ERP29, GTPB6, HSPB1, HUWE1, MPV17L2, PSMA7, SEC61 G, TOMM22, UBE2B, UBE2Q1, USP14, USP16 or USP45 gene.

36. The method of claim 35, wherein the agent is a small molecule inhibitor or a monoclonal antibody.

37. (canceled)

38. The method of claim 31, wherein the mutant TPI protein comprises:
a glutamic acid to aspartic acid substitution at position 105 (TPI^{E105D});
a valine to methionine substitution at position 232 (TPI^{V232M});
a glutamine to proline substitution at position 181 (TPI^{Q181P});
an arginine to glutamine mutation at position 190 (TPI^{R190Q});
a cysteine to tyrosine substitution at position 42 (TPI^{C42Y});
an alanine to aspartic acid substitution at position 63 (TPI^{A63D});
a glycine to alanine substitution at position 73 (TPI^{G73A});
a glycine to arginine substitution at position 123 (TPI^{G123R});
a valine to methionine substitution at position 155 (TPI^{V155M});
an isoleucine to valine substitution at position 171 (TPI^{I171V});
a phenylalanine to leucine substitution at position 241 (TPI^{F241E}); or
a phenylalanine to serine substitution at position 241 (TPI^{E241S}),
wherein amino acid numbering is based on SEQ ID NO: 1.

39-42. (canceled)


SEQUENCE LISTING (1 sequence)

SEQ ID NO: 1 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ